(19) United States
(12) Patent Application Publication
(10) Pub. No.: US 2022/0062142 A1
(43) Pub. Date: Mar. 3, 2022

(54) HAIR LIGHTENING COMPOSITIONS PROVIDING DAMAGE PROTECTION AND SENSORIAL BENEFITS AND METHODS OF USE

(71) Applicant: L'OREAL, Paris (FR)

(72) Inventor: Derek HENRY, Montclair, NJ (US)

(73) Assignee: L'OREAL, Paris (FR)

(21) Appl. No.: 17/003,558

(22) Filed: Aug. 26, 2020

(51) Int. Cl. A61K 8/60; A61K 8/362; A61K 8/9767; A61K 8/44; A61K 8/92; A61Q 5/08

(52) U.S. Cl. CPC A61K 8/602; A61K 8/362; A61K 8/9767; A61Q 5/08; A61K 8/925; A61K 8/922; A61K 8/92; A61K 8/44

(57) ABSTRACT

Hair treatment and hair lightening compositions, methods of making and use, and to kits comprising the compositions. The components of the hair treatment compositions may be included in hair lightening compositions and used to lighten and/or color hair. The hair treatment compositions include: (a) one or more polycarboxylic acids, and/or a salt thereof; (b) one or more amino acids, and/or a salt thereof; and (c) one or more antioxidant comprising an aromatic ring having two alcohol functional groups. The hair lightening compositions further include (d) one or more bleaching agents.

### FIELD OF THE DISCLOSURE

The present disclosure relates to compositions and methods for treating hair, kits comprising the compositions, and methods for using the compositions.

### BACKGROUND

Cosmetic care compositions are frequently utilized by consumers in order to enhance, treat or change the appearance of keratinous substrates, such as hair, such as for example by changing the color, style, and/or shape of the hair, and/or by imparting various properties to hair, such as shine and conditioning. Many of the known compositions and processes for enhancing the appearance of the hair involve chemical treatment of the hair.

For example, one such chemical treatment of hair involves changing the color. This may involve removing color and/or depositing an artificial color onto the hair to provide a different shade or color, and/or to lift the color of the hair during lightening of darker hair to lighter shades. The process of lifting the color of hair, also known as lightening, generally requires the use of compositions having at least one oxidizing agent. Variation of tone height before and after the application of a hair color-altering composition is used to evaluate lightening or lifting of the color of the hair. Degree or level of lighting or lift corresponds to the variation. Notion of “tone” is based on the classification of the natural shades, one tone separating each shade from the shade immediately following or preceding it, which is well known to hairstyling professionals. Tone heights or levels range from 1 (black) to 10 (light blond), one unit corresponding to one tone. Thus, the higher the number, the lighter the shade or the greater the degree of lift.

Hair lightening or color lifting compositions and hair dyeing compositions are typically alkaline, having a pH value above 7, and generally being at pH 9 and above. Presence of an alkalizing agent in amounts sufficient to make such compositions alkaline is generally required, such as ammonia or an ammonia gas generating compound and/or an amine or ammonium-based compound. Alkalizing agent causes the hair shaft to swell, thus allowing the small oxidative dye molecules to penetrate the cuticle and cortex of the hair before the oxidation condensation process is completed. Resulting larger-sized colored complexes from the oxidative reaction are then trapped inside the hair fiber, thereby permanently altering the color of the hair.

Additionally, there are many techniques and compositions for styling or altering the shape of hair. For example, hair care products referred to as “hair relaxers” or “hair straighteners” can relax or straighten curly or kinky hair, including wavy hair. Straightening or relaxing the curls of very curly hair may increase the manageability and ease of styling of such hair. Compositions for permanent waving the hair will impart a curl or a wave to otherwise straight hair. Different types of compositions can be applied onto hair in order to change its shape and make it more manageable, such as alkaline and acidic compositions. Hair relaxers, straighteners, perms, and/or waves may either be applied in a hair salon by a professional or in the home by the individual consumer.

These chemical treatments can damage the hair fibers and/or irritate the scalp while dyeing and/or lightening, relaxing, straightening, perming, and/or waving the hair. Thus, in order to reduce, or avoid, damaging the hair, as well as to improve the cosmetic performance of the compositions, the use of new, additional components and/or novel combinations of ingredients are continuously sought.

Various compositions have been provided, however, frequently the choice of components or combinations of ingredients pose difficulties insofar as they often are unstable, detrimental to other cosmetic attributes and/or present other problems. For example, many of the heretofore and utilized compositions lack ease and uniformity of application, and/or have poor rheology, viscosity, stability, color deposit and/or target shade formation. As a result, these compositions yield more disadvantages, and in many cases lead to increased damage and/or a less healthy look to the hair. It would therefore be desirable to provide the consumer with compositions and methods that can treat the hair, e.g. lift the color of hair and/or deposit color onto hair in an efficient manner, while providing other cosmetic advantages such as shine, conditioning, fiber strength, and/or a healthy appearance to the hair, but avoiding or minimizing damage to the hair.

Moreover, typically both natural and sensitized and /or chemically treated hair can contain several kinds of negatively charged moieties, for example, carboxylates (resulting from the hydrolysis of amino acids and thioester bonds) and/or sulfonates (resulting from the oxidation of disulfide bonds). These negatively charged moieties can degrade the cosmetic properties of the hair. Additionally, when hair is chemically treated or damaged, the disulfide bonds in hair (disulfide linkages between two cysteine units) can be reduced or broken, resulting in the formation of thiol groups and/or cysteic acid.

Thus, there is a need in the art for novel compositions that protect hair fibers during bleaching, thereby reducing damage or further damage to the hair. There is a further need in the art for compositions that impart cosmetic improvement and improved feel to bleached hair, such as strengthening, enhanced softness, shine, conditioning, and healthy appearance, during hair coloring, lightening, straightening, relaxing, and/or shaping.

### SUMMARY OF THE DISCLOSURE

The present disclosure relates to compositions and methods for reducing damage done to hair fibers during bleaching, while also providing treatment and cosmetic improvement to hair fibers compared to traditional post-bleach feel of the hair fibers. Uniquely, the subject compositions and methods, provide stable compositions with built-in antioxidant and bonder properties that dramatically improve the quality and durability of hair during hair lightening. Damage during chemical treatment (lightening/bleaching process) is minimized and/or treated due to the use of the subject composition, which, has been found to protect the keratin fibers of the hair.

The subject compositions utilize one or more antioxidant having an aromatic ring with two alcohol functional groups, preferably baicalin, in combination with a polycarboxylic acid and an amino acid built-into a hair lightening composition to render a built-in bleach system that reduces damage to the hair. It has been surprisingly and unexpectedly found that the antioxidant with an aromatic ring with two alcohol functional groups, a polycarboxylic acid and amino acid combination is stable in a powder form when mixed with bleach powder/hair color altering agent/lightening agent, providing a built-in one-step bleaching process. Furthermore, it has been surprisingly and unexpectedly found that the combination of the one or more antioxidant having an aromatic ring with two alcohol functional groups, polycarboxylic acid and an amino acid combination provides significant benefits to fiber integrity during hair bleaching, resulting in improved tensile strength and elastic modulus, and reduced formation of citric acid, when compared to the same bleach without the required combination. Additionally, it has been found that the subject composition provides lasting smoothness and manageability to the hair after bleaching, which, on information and belief, is directly tied to the presence of the aromatic ring with two alcohol functional groups in the antioxidant synergistically operating in combination with the polycarboxylic acid and amino acid.

The invention provides, in various embodiments, compositions and kits formulated for various modes of delivery to a keratinous material to one or more of: confer protection to the keratinous material from the damaging effects of oxidants and oxidizing agents in the color altering system; enhance and protect one or more sensorial features of the keratinous material treated with oxidizing agents in the color altering system; and protect and preserve the mechanical properties and native amino acid structure of the hair.

The subject invention is directed to a built-in bleach composition with one or more selected antioxidant with an aromatic ring with two alcohol functional groups in combination with a polycarboxylic acid and an amino acid. Non-limiting benefits of the subject built-in bleach system include that the antioxidant, polycarboxylic acid and an amino acid system is stable in a powder form when mixed with bleach powder, providing a one-step bleaching process. The antioxidant, polycarboxylic acid and an amino acid combination has been found to provide benefits to fiber integrity during hair bleaching, resulting in improved tensile strength and elastic modulus, and reduced formation of citric acid, as compared to bleach compositions without the novel combination. Additionally, the subject composition has been found to provide lasting smoothness and manageability to the hair after bleaching, which on information and belief, and without being bound by theory, is directly tied to the presence of the aromatic ring with two alcohol functional groups in the antioxidant.

In one embodiment, the antioxidant comprises at least one antioxidant having an aromatic ring with two alcohol functional groups. Preferably the antioxidant comprises at least one polyphenol selected from baicalin, ferulic acid, pine bark extract, polydatin and ellagic acid. Most preferably, the antioxidant may comprise at least one polyphenol having an aromatic ring with two alcohol functional groups selected from a flavonoid glycoside, optionally comprising baicalin.

In the various embodiments, one or more of each of the antioxidant and the components of the color altering system may be provided separately packaged, or packaged in combinations, such as for example a combination of an antioxidant selected from a polyphenol having an aromatic ring with two alcohol functional groups and a bleach and a developer, or in another example, a combination of an antioxidant and a bleach, or in another example a combination of an antioxidant and a colorant, and in yet another example, a combination of an antioxidant and a developer. According to such various embodiments, the various separately packaged or combined components that include an antioxidant, a bleach or colorant, and a developer, may be mixed prior to application to a keratinous material, or may be individually applied to the keratinous material in a particular sequence or order, or at the same time.

Thus, in some embodiments, the invention provides a treatment composition comprising (a) an antioxidant composition comprising at least one antioxidant compound selected from a polyphenol having an aromatic ring with two alcohol functional groups, and a color altering system including at least one of (b) a bleach, (c) a developer, and (d), a colorant which may be selected from oxidative dye precursors, wherein, according to methods of use of the composition, (a) and one or more of (b) and (c), or (a) and one or more of (c) and (d) are combined or combinable by one or more of sequential application to a keratinous material or premixing followed by application to a keratinous material.

According to some embodiments, the at least one antioxidant compound is selected from polyphenols, chosen from baicalin, ferulic acid, pine bark extract, polydatin and ellagic acid. In accordance with the various embodiments, the antioxidant composition comprising the at least one antioxidant compound includes one or more auxiliaries or additives.

The treatment compositions of the instant disclosure typically include (a) one or more polycarboxylic acids, and/or a salt thereof; (b) one or more amino acids, and/or a salt thereof; and (c) one or more antioxidant comprising a polyphenol having an aromatic ring with two alcohol functional groups. The treatment compositions may be separate compositions that are combined with one or more bleaching agents prior to use, or, preferably, can be part of a single built-in hair lightening composition (a composition having one or more bleaching agents). The hair treatment compositions optionally include \ one or more color altering system component. The at least one color altering system component is preferably selected from one or more of: a bleaching agent; a developer; a colorant selected from oxidative dye precursors and direct dyes; a bleach and a developer; a colorant selected from oxidative dye precursors and direct dyes and a developer; and mixtures thereof.

Useful polycarboxylic acids include, but are not limited to, dicarboxylic acids and/or tricarboxylic acids, a salt thereof, and a mixture thereof. Non-limiting of dicarboxylic acids include oxalic acid, malonic acid, malic acid, glutaric acid, citraconic acid, succinic acid, adipic acid, tartaric acid, fumaric acid, maleic acid, sebacic acid, azelaic acid, dodecanedioic acid, phthalic acid, isophthalic acid, terephthalic acid, and 2,6-naphthalene dicarboxylic acid, a salt thereof, and a mixture thereof. In some cases, particularly useful dicarboxylic acids include malonic acid and/or maleic acid, and/or salts thereof. Non-limiting examples of tricarboxylic acids include citric acid, isocitric acid, trimesic acid, aconitric acid (cis and/or trans), and propane-1,2,3-tricarboxylic acid, and salts thereof. In some cases, a particularly useful tricarboxylic acid is citric acid, and/or a salt thereof. Preferably, the one or more polycarboxylic acid comprises dicarboxylic acids selected from maleic acid, malonic acid, citric acid, a salt thereof, and/or a mixture thereof.

Useful amino acids include, but are not limited to, aliphatic amino acids such as glycine, alanine, valine, leucine, and isoleucine, and salts thereof. In some cases, glycine and/or a salt thereof is particularly useful.

Useful cationic conditioning agents, if present, include, but are not limited to, monoalkyl quaternary amines, dialkyl quaternary amines, and polyquaternium compounds.

The instant disclosure relates to hair lightening compositions that include the components of the hair treatment compositions mentioned above. For instance, the instant disclosure relates to hair lightening compositions comprising: (a) one or more polycarboxylic acids, and/or a salt thereof; (b) one or more amino acids, and/or a salt thereof; (c) one or more antioxidants comprising polyphenol having an aromatic ring with two alcohol functional groups; and (d) one or more bleaching agents. Non-limiting examples of polycarboxylic acids, amino acids, and antioxidants are provided above.

Bleaching agents may include, but are not limited to peroxides, persulfates, perborates percarbonates, alkali metal bromates, ferricyanides, peroxygenated salts, and mixtures thereof.

In some cases, the hair lightening compositions further include one or more oils, which may be one or more ester oils, silicone oils, fluoro oils, vegetable oils, animal oils, hydrocarbon-based oils, and mixtures thereof. Additionally, the hair lightening compositions may optionally include one or more thickening agents. Useful thickening agent include, but are not limited to, carboxylic acid polymers, crosslinked polyacrylate polymers, polyacrylamide polymers, polysaccharides, and gums, in particular guar gum.

The hair treatment compositions and/or the hair lightening compositions are often anhydrous or essentially anhydrous.

The hair lightening composition may optionally include colorants and/dyes. Useful colorants are those that are stable in the hair lightening compositions. These colorants can be used, for example, to impart toning and coloring to hair. Non-limiting colorants include pigments, liposoluble dyes, direct dyes, oxidative dye precursors, nacreous pigments, pearling agents, leuco dyes, optical lightening colorants, natural colorants and optically-variable pigments. Couplers may also be incorporated into the hair lightening compositions.

In another instance, a hair lightening composition is provided. The hair lightening composition comprises one or more polycarboxylic acid and/or a salt thereof; one or more amino acid, and/or a salt thereof; one or more antioxidant; and one or more bleaching agent. Preferably, the one or more polycarboxylic acid is selected from one or more dicarboxylic acids, one or more tricarboxylic acids, a salt thereof, and/or a mixture thereof. The one or more dicarboxylic acids may be selected from oxalic acid, malonic acid, malic acid, glutaric acid, citraconic acid, succinic acid, adipic acid, tartaric acid, fumaric acid, maleic acid, sebacic acid, azelaic acid, dodecanedioic acid, phthalic acid, isophthalic acid, terephthalic acid, 2,6-naphthalene dicarboxylic acid, a salt thereof, and a mixture thereof; and the one or more tricarboxylic acids are selected from the group consisting of citric acid, isocitric acid, trimesic acid, aconitric acid (cis and/or trans), and propane-1,2,3-tricarboxylic acid, a salt thereof, and a mixture thereof. Preferably, the one or more amino acids comprises one or more aliphatic amino acids selected from glycine, alanine, valine, leucine, isoleucine, a salt thereof, and a mixture thereof. The one or more antioxidant preferably comprises one or more polyphenol selected from baicalin, ferulic acid, pine bark extract, polydatin, ellagic acid, and mixtures thereof.

In another aspect, a hair lightening composition is provided comprising: about 0.1 to about 5 wt. % of one or more polycarboxylic acid selected from maleic acid, malonic acid, citric acid, a salt thereof, or a mixture thereof; about 0.01 to about 5 wt. %

of one or more aliphatic amino acid selected from glycine, alanine, valine, leucine, isoleucine, a salt thereof, and a mixture thereof; about 0.01 to about 5 wt. % of one or more antioxidant comprising an aromatic ring having two alcohol functional groups; about 20 to about 80 wt. % of one or more persulfates selected from the group consisting of sodium persulfate, potassium persulfate, ammonium persulfate, and a mixture thereof; about 0.1 to about 10 wt. % of one or more oils; about 0.1 to about 10 wt. % of one or more thickening agents; about 0.1 to about 10 wt. % of one or more anionic surfactants; and about 1 to about 40 wt. % of one or more alkalizing agents selected from the group consisting of ammonium hydroxide, sodium silicate, sodium metasilicate, monoethanolamine, and a mixture thereof, wherein the wt. % in all instances is based on the total weight of the hair lightening composition.

The instant disclosure additionally relates to kits comprising the hair treatment compositions and/or hair lightening compositions. For example, kits can include a hair treatment composition that is separate from a hair lightening composition. The kits can optionally include an aqueous developer composition (often comprising a peroxide such as hydrogen peroxide). Alternatively, kits can include a hair lightening composition of the instant disclosure (a single compositions including the components of the treatment compositions and bleaching agent(s)) and include a separate aqueous developer composition. In one aspect, the kit comprises a hair lightening composition including: (a) one or more polycarboxylic acid and/or a salt thereof; (b) one or more amino acid, and/or a salt thereof; (c) one or more antioxidant comprising an aromatic ring having two alcohol functional groups; and (d) one or more bleaching agent; an aqueous developer composition comprising one or more peroxides; and optionally, a third composition comprising one or more hair conditioning agents and/or one or more direct dyes. Typically, the aqueous developer compositions include one or more peroxides, such as hydrogen peroxide. Optionally, the kits may include an additional composition comprising one or more hair conditioning agents and/or one or one or more hair coloring agents.

Finally, the instant disclosure relates to methods for lightening and/or coloring hair; and to methods for protecting and/or improving the appearance of hair, wherein the methods involve application of a composition described herein to the hair; allowing the composition to remain on the hair for a sufficient amount of time for processing (about 1 min. to about 60 min.); and rinsing the compositions from the hair. One aspect of the invention provides a method for lightening hair comprising: mixing a hair lightening composition with an aqueous developer composition comprising one or more peroxides, the hair lightening composition comprising: (a) one or more polycarboxylic acid and/or a salt thereof; (b) one or more amino acid, and/or a salt thereof; (c) one or more antioxidant comprising an aromatic ring having two alcohol functional groups; (d) one or more bleaching agent; applying the mixture onto hair and allowing the mixture to remain on the hair for about 1 to about 45 minutes; and rinsing the mixture from hair.

### DETAILED DESCRIPTION OF THE DISCLOSURE

The instant disclosure relates to hair care compositions that are useful for lightening and/or coloring hair. The compositions are unique in that they reduce the amount of damage caused to the hair due to chemical processing (bleaching). A hair treatment composition relates to a composition that strengthens hair and/or reduces the damage incurred to the hair by chemical processing including the bleaching and/or coloring of the hair. Treated hair fibers (hair treated with the compositions) have desirable cosmetic properties such as improved gloss, improved combability, and improved strength and elasticity. Successful treatment of the hair can be determined by, for example, by cystic acid analysis.

It has been surprisingly and unexpectedly discovered by the inventors that compositions comprising a combination of one or more polycarboxylic acid, one or more amino acid and one or more antioxidant comprising an aromatic ring having two alcohol functional groups utilized in combination with a color altering system, such as an oxidizing lifting or bleaching or coloring composition, when applied to keratinous materials such as hair, with and without heat, confer protection to the keratinous material from the damaging effects of bleaching and/or of oxidizing agents in the color altering system; enhance and protect one or more sensorial features and preserve the mechanical properties and native amino acid structure of the keratinous material treated with bleaching and/or oxidizing agents in the color altering system. Thus, the inventive treatment compositions confer protection to the keratinous material from the damaging effects of bleaching and/or oxidizing agents to enhance and protect one or more sensorial features selected from combability, softness, elasticity, moisture and shine. In some particular examples, the inventors have discovered that using the polycarboxylic acid, amino acid and antioxidant composition on hair prior to or contemporaneously at the time of application of at least a bleach (oxidative precursor), particularly an antioxidant comprising an aromatic ring having two alcohol functional groups selected from flavonoid glycosides, preferably baicalin, and/or other polyphenols, resulted in significant retention of mechanical and sensorial properties of the hair as compared with treatments with color altering systems in the absence of an antioxidant.

Without being bound to any one theory, the inventors of the present disclosure believe that the polycarboxylic acid, amino acid and antioxidant compounds in the treatment compositions of the invention synergistically serve to capture oxidative species (free radicals) generated in the oxidation reaction to prevent their association with the keratin fibers, thereby protecting the keratinous material from damage or minimizing damage to hair. Thus, while other compositions that employ acid may effectively reduce pH and slow down the rate of oxidation, they lack the benefit of protecting the keratin fibers from damage or minimizing damage caused by excess radicals. The inventive compositions provide a surprising and unexpected protective benefit that is not observed with comparative formulations that comprise acids.

The compositions, according to the invention, are compositions including one or more each of polycarboxylic acid, amino acid and antioxidant compounds, and components of a bleach and/or color altering system, including optionally one or more dyes or colorants. The composition may include other suitable ingredients for hair treatment or hair repair. For example, known solvents and/or additives may be utilized in addition to the polycarboxylic acid, amino acid and antioxidant composition to provide additional benefits to the composition.

The hair treatment compositions of the instant disclosure typically include:
(a) one or more polycarboxylic acids and/or a salt thereof;
(b) one or more amino acids, and/or a salt thereof; and
(c) one or more antioxidant comprising an aromatic ring having two alcohol functional groups.

The amounts of the three components of the hair treatment compositions can vary and will depend on the other components (and the amounts of the other components) in the hair treatment compositions, if present. Nonetheless, in general, regardless of the other components in the hair treatment composition, the ratio of the (a) one or more polycarboxylic acids to the (b) one or more amino acids is about 2:1 to about 10:1. Furthermore, the ratio of the (a) one or more polycarboxylic acids to the (b) one or more amino acids may be about 3:1 to about 9:1, about 4:1 to about 8:1, or about 5:1 to about 6:1.

In general, the ratio of the (a) one or more polycarboxylic acids to the (c) one or more antioxidant is about 1:1 to about 8:1. However, the ratio of the (a) one or more polycarboxylic acids to the (c) one or more antioxidants may be about 2:1 to about 7:1, about 2:1 to about 6:1, about 2:1 to about 5:1, about 3:1 to about 7:1, about 3:1 to about 6:1, about 3:1 to about 5:1, or about 3:1 to about 4:1.

In general, the ratio of the (a) one or more polycarboxylic acids to the (c) one or more antioxidant is about 1:1 to about 8:1, if one or more cationic conditioning agents are present. However, the ratio of the (a) one or more polycarboxylic acids to the (c) one or more cationic conditioning agents may be about 2:1 to about 7:1, about 2:1 to about 6:1, about 2:1 to about 5:1, about 3:1 to about 7:1, about 3:1 to about 6:1, about 3:1 to about 5:1, or about 3:1 to about 4:1.

Generally, the ratio of the (b) one or more amino acids to the (c) one or more antioxidant is about 1:1 to about 1:10. Furthermore, the ratio of the (b) one or more amino acids to the (c) one or more antioxidant may be about 1:2 to about 1:9, about 1:4 to about 1:8, or about 1:5 to about 1:6.

In general, the ratio of the (b) one or more amino acids to the (c) one or more cationic conditioning agents is 0.1:1 to about 5:1, if present. However, the ratio of the (b) one or more amino acids to the (c) one or more cationic conditioning agents may be about 0.1:1 to about 2:1, about 0.1:1 to about 1:1, about 0.2:1 to about 3:1, about 0.2:1 to about 2:1, about 0.2:1 to about 1:1, about 0.5:1 to about 3:1, about 0.5:1 to about 2:1, or about 0.5:1 to about 1:1.

The instant disclosure also relates to hair lightening compositions that include the components of the hair treatment compositions mentioned above. A treatment composition can be added directly to a hair lightening composition to derive a ready-to-use hair lightening composition. For example, the hair treatment compositions can be added to a hair lightening composition, mixed, and the mixture immediately (within 1 to 60 minutes) applied to hair. Alternatively, the hair lightening composition can already include the components of the hair treatment composition (no separate packing and mixing is required). Accordingly, the instant disclosure relates to hair lightening compositions comprising:
(a) one or more polycarboxylic acids, and/or a salt thereof;
(b) one or more amino acids, and/or a salt thereof;
(c) one or more antioxidant; and
(d) one or more bleaching agents.

Optionally, the composition may further comprise one or more cationic conditioning agents.

The ratios for components (a), (b), and (c) discussed above with respect to the treatment compositions can apply to the hair lightening compositions.

In some cases, the one or more polycarboxylic acids are di-, tri-, and/or tetra-carboxylic acids. Non-limiting examples of dicarboxylic acids include oxalic acid, malonic acid, malic acid, glutaric acid, citraconic acid, succinic acid, adipic acid, tartaric acid, fumaric acid, maleic acid, sebacic acid, azelaic acid, dodecanedioic acid, phthalic acid, isophthalic acid, terephthalic acid, and 2,6-naphthalene dicarboxylic acid, and salts thereof. Non-limiting examples of tricarboxylic acids include citric acid, isocitric acid, trimesic acid, aconitric acid (cis and/or trans), and propane-1,2,3-tricarboxylic acid, and salts thereof. A non-limiting example of a tetracarboxylic acid is butane tertracarboxylic acid, and a salt thereof. In some instances, the polycarboxylic acid may be selected from the group consisting of maleic acid, malonic acid, citric acid, a salt thereof, and a mixture thereof.

The total amount of the one or more polycarboxylic acids, and/or salt thereof, can vary, but is typically about 0.1 to about 10 wt. %, based on the total weight of the hair lightening composition. In some cases, the total amount of the one or more polycarboxylic acids, and/or salt thereof is from about 0.1 to about 8 wt. %, about 0.1 to about 6 wt. %, about 0.1 to about 5 wt. %, about 0.5 to about 10 wt. %, 0.5 to about 8 wt. %, about 0.5 to about 6 wt. %, about 0.5 to about 5 wt. %, about 1 to about 10 wt. %, about 1 to about 8 wt. %, about 1 to about 6 wt. %, or about 1 to about 5 wt. %.

Amino acids are well known. An “amino acid” is any organic compound having both an amino group and a carboxylic acid group. In some cases, the one or more amino acids are aliphatic amino acids, for example, those selected from the group consisting of glycine, alanine, valine, leucine, isoleucine, a salt thereof, and a mixture thereof. Glycine and/or a salt of glycine is particularly useful in some instances. The total amount of the one or more amino acids, and/or salts thereof, can vary but typically the hair lightening compositions comprise about 0.01 to about 5 wt. % of the one or more amino acids, and/or salt thereof, based on the total weight of the hair lightening composition. In some cases, the total amount of the one or more amino acids and/or salts thereof, is about 0.01 to about 4 wt. %, about 0.01 to about 3 wt. %, about 0.01 to about 2 wt. %, about 0.1 to about 5 wt. %, about 0.1 to about 4 wt. %, about 0.1 to about 3 wt. %, or about 0.1 to about 2 wt. %.

The compositions comprise one or more antioxidant compound. Some representative examples of antioxidant compounds include baicalin (Scutellaria Baicalensis root extract), pine bark extract (Pinus Pinaster bark/bud extract), ferulic acid (a hydroxycinnamic acid) and ellagic acid. Other examples include dihydrochalone, ascorbic acid, zinc PCA, and polydatin. More generally, the antioxidant compounds may be selected from among other phenolic compounds (including polyphenols).

Optionally, the compositions may contain one or more cationic conditioning agents are well known. Non-limiting examples of the one or more cationic conditioning agents useable in the compositions of the instant disclosure include monoalkyl quaternary amines, dialkyl quaternary amines, and polyquaternium compounds. In some cases, polyquaternium compounds are used and in some cases the polyquaternium compounds are polyquaternium polymers. One type of polyquaternium polymers that are useful include quaternized copolymer(s) of dimethyldiallylammonium and acrylic acid, such as for example, polyquaternium-22. The total amount of the one or more cationic conditioning agents may vary but when present are typically in an amount of about 0.01 to about 10 wt. %, based on the total weight of the hair lighting composition. In some cases, the total amount of the one or more cationic conditioning agents is about 0.01 to about 8 wt. %, about 0.01 to about 6 wt. %, about 0.01 to about 5 wt. %, 0.01 to about 4 wt. %, about 0.01 to about 3 wt. %, about 0.1 to about 10 wt. %, 0.1 to about 8 wt. %, about 0.1 to about 6 wt. %, about 0.1 to about 5 wt. %, about 0.1 to about 4 wt. %, about 0.1 to about 3 wt. %, about 0.5 to about 10 wt. %, about 0.5 to about 8 wt. %, about 0.5 to about 6 wt. %, about 0.5 to about 5 wt. %, about 0.5 to about 4 wt. %, or about 0.5 to about 3 wt. %.

Bleaching agents are well known. Non-limiting examples of bleaching agents include peroxides, persulfates, perborates percarbonates, alkali metal bromates, ferricyanides, peroxygenated salts, and mixtures thereof. Bleaching agents that may also be used include at least one redox enzyme such as laccases, peroxidases, and 2-electron oxidoreductases, such as uricase, where appropriate in the presence of their respective donor or co-factor. In some cases, the one or more bleaching agents are persulfates, for example, one or more of sodium persulfate, potassium persulfate, and ammonium persulfate. The total amount of the one or more bleaching agents can vary but is typically about 20 to about 80 wt. %, based on the total weight of the hair lightening composition. In some cases, the total amount of the bleaching agent may be from about 20 to about 70 wt. %, about 20 to about 60 wt. %, about 30 to about 80 wt. %, about 30 to about 70 wt. %, about 30 to about 60 wt. %, about 40 to about 80 wt. %, about 40 to about 70 wt. %, about 40 to about 60 wt. %, about 50 to about 80 wt. %, about 50 to about 70 wt. %, or about 50 to about 60 wt. %.

In some cases, the hair lightening compositions include one or more oils. Non-limiting examples of oils include ester oils, silicone oils, fluoro oils, vegetable oils, animal oils, hydrocarbon-based oils, and mixtures thereof. In some instances, hydrocarbon-based oils, such as mineral oil, can be included. The total amount of the one or more oils can vary but is typically about 0.1 to about 10 wt. %, based on the total weight of the hair lightening composition. In some cases, the total amount of the one or more oils is about 0.1 to about 8 wt. %, about 0.1 to about 6 wt. %, about 0.1 to about 5 wt. %, about 0.5 to about 10 wt. %, about 0.5 to about 8 wt. %, about 0.5 to about 5 wt. %, about 1 to about 10 wt. %, about 1 to about 8 wt. %, about 1 to about 6 wt. %, or about 1 to about 5 wt. %.

The hair lightening composition can also optionally include one or more thickening agents. Thickening agents are well known. Nonetheless, non-limiting examples of thickening agents include carboxylic acid polymers, crosslinked polyacrylate polymers, polyacrylamide polymers, polysaccharides, and gums. More specific, non-limiting examples of thickening agents include acacia, agar, algin, alginic acid, ammonium alginate, amylopectin, calcium alginate, calcium carrageenan, carnitine, carrageenan, dextrin, gelatin, gellan gum, guar gum, guar hydroxypropyltrimonium chloride, hydroxylpropyl guar hydroxypropyltrimonium chloride, hectorite, hyaluroinic acid, hydrated silica, hydroxypropyl chitosan, hydroxypropyl guar, karaya gum, kelp, locust bean gum, natto gum, potassium alginate, potassium carrageenan, propylene glycol alginate, sclerotium gum, sodium carboyxmethyl dextran, sodium carrageenan, tragacanth gum, xanthan gum, and mixtures thereof. In some cases, the one or more thickening agents include a crosslinked polyacrylate polymer, for example, acrylates/C10-30 alkylacrylate crosspolymer. Furthermore, in some cases the one or more thickening agents include guar gum.

The total amount of the one or more thickening agents can vary but is typically about 0.1 to about 10 wt. %, based on the total weight of the hair lightening composition. In some cases, the total amount of the one or more thickening agents is about 0.01 to about 8 wt. %, about 0.01 to about 6 wt. %, about 0.01 to about 5 wt. %, 0.01 to about 4 wt. %, about 0.01 to about 3 wt. %, about 0.1 to about 10 wt. %, 0.1 to about 8 wt. %, about 0.1 to about 6 wt. %, about 0.1 to about 5 wt. %, about 0.1 to about 4 wt. %, about 0.1 to about 3 wt. %, about 0.5 to about 10 wt. %, about 0.5 to about 8 wt. %, about 0.5 to about 6 wt. %, about 0.5 to about 5 wt. %, about 0.5 to about 4 wt. %, or about 0.5 to about 3 wt. %.

The hair lightening compositions may include one or more anionic surfactants. For instance, the anionic surfactant(s) that may be useful include alkyl sulfates, alkyl ether sulfates, alkylamido ether sulfates, alkylaryl polyether sulfates, monoglyceride sulfates, alkylsulfonates, alkylamide sulfonates, alkylarylsulfonates, alpha-olefin sulfonates, paraffin sulfonates, alkylsulfosuccinates, alkyl ether sulfosuccinates, alkylamide sulfosuccinates, alkyl sulfoacetates, acylsarcosinates, acylglutamates, alkylsulfosuccinamates, acyl isethionates and N-acyltaurates, salts of alkyl monoesters and polyglycoside-polycarboxylic acids, acyllactylates, salts of D-galactoside uronic acids, salts of alkyl ether carboxylic acids, salts of alkyl aryl ether carboxylic acids, and salts of alkylamido ether carboxylic acids; or the non-salified forms of all of these compounds, the alkyl and acyl groups of all of these compounds containing from 6 to 24 carbon atoms and the aryl group denoting a phenyl group. Some of these compounds may be oxyethylenated and then preferably comprise from 1 to 50 ethylene oxide units.

The salts of C₆-C₂₄ alkyl monoesters of polyglycoside-polycarboxylic acids may be chosen from C₆-C₂₄ alkyl polyglycoside-citrates, C₆-C₂₄ alkyl polyglycoside-tartrates and C₆-C₂₄ alkyl polyglycoside-sulfo succinates.

When the anionic surfactant(s) are in salt form, they may be chosen especially from alkali metal salts such as the sodium or potassium salt and preferably the sodium salt, ammonium salts, amine salts, and in particular amino alcohol salts, or alkaline-earth metal salts such as the magnesium salt. Examples of amino alcohol salts that may especially be mentioned include monoethanolamine, diethanolamine and triethanolamine salts, monoisopropanolamine, diisopropanolamine or triisopropanolamine salts, 2-amino-2-methyl-1-propanol salts, 2-amino-2-methyl-1,3-propanediol salts and tris(hydroxymethyl)aminomethane salts. Alkali metal or alkaline-earth metal salts and in particular the sodium or magnesium salts may be used.

Use is also made of (C₆-C₂₄)alkyl sulfates, (C₆-C₂₄)alkyl ether sulfates, which are optionally ethoxylated, comprising from 2 to 50 ethylene oxide units, and mixtures thereof, in particular in the form of alkali metal salts or alkaline-earth metal salts, ammonium salts or amino alcohol salts.

In some cases, the one or more anionic surfactant is selected from the group consisting of sodium oleyl succinate, ammonium lauryl sulphosuccinate, sodium lauryl sulfate, sodium lauryl ether sulfate (also known as sodium laureth sulfate, SLES), sodium lauryl ether sulphosuccinate, ammonium lauryl sulfate (ALS), ammonium lauryl ether sulfate (ammonium laureth sulfate), sodium dodecylbenzene sulphonate, triethanolamine dodecylbenzene sulphonate, sodium cocoyl isethionate, sodium lauryl isethionate, lauryl ether carboxylic acid, sodium N-lauryl sarcosinate, and a mixture thereof. In some instances, preferred anionic surfactants include sodium lauryl sulfate, sodium lauryl ether sulfate, sodium lauryl ether sulphosuccinate, ammonium lauryl sulfate, ammonium lauryl ether sulfate, or a mixture thereof.

The total amount of the one or more anionic surfactants is typically about 0.1 to about 10 wt. %, based on the total weight of the hair lightening composition. In some cases, the total amount of the one or more anionic surfactants is about 0.1 to about 8 wt. %, about 0.1 to about 6 wt. %, about 0.1 to about 5 wt. %, about 0.1 to about 3 wt. %, or about 0.5 to about 2 wt. %, based on the total weight of the hair lightening composition.

The hair lightening compositions often include one or more alkalizing agents. The term “alkalizing agent” means an ingredient that is capable imparting alkalinity (e.g. a pH of greater than 7) to the hair lightening composition. The hair lightening compositions typically have an alkaline pH (a pH of greater than 7), e.g., a pH of about 8 to about 11, or about 8 to about 10. Suitable alkalizing agents include ammonium hydroxide, metal hydroxides, alkanolamines, sodium silicate, metal carbonates, such as magnesium carbonate, sodium metasilicate, and a mixture thereof. Suitable metal hydroxides and carbonates include alkali metal and alkaline earth metal hydroxides or carbonates. Examples of such metal hydroxides include sodium, potassium, lithium, calcium, magnesium and so on. A common alkaline earth metal hydroxide is sodium hydroxide. Suitable alkanolamines include mono-, di-, and trialkanolamines such as monoethanolamine (MEA), diethanolamine (DEA), triethanolamine (TEA), 2-aminobutanol, aminoethyl propanediol, aminomethyl propanediol, bis-hydroxyethyl tromethamine, diethanolamine, diethyl ethanolamine, diisopropanolamine, dimethylamino methylpropanol, dimethyl MEA, isopropanolamine, methylethanolamine, mixed isopropanolamines, triisopropanolamine, tromethamine, and mixtures thereof. A particularly preferred alkanolamine is MEA. In some instances, the hair lightening compositions include one or more alkalizing agents selected from the group consisting of ammonium hydroxide, sodium silicate, sodium metasilicate, monoethanolamine, and a mixture thereof.

The total amount of the one or more alkalizing agents can vary but is typically about 1 to about 40 wt. %, based on the total weight of the hair lightening composition.

In some instances, the total amount of the one or more alkalizing agents is about 1 to about 35 wt. %, about 1 to about 30 wt. %, about 1 to about 25 wt. %, about 5 to about 40 wt. %, about 5 to about 35 wt. %, about 5 to about 30 wt. %, about 5 to about 25 wt. %, about 10 to about 40 wt. %, about 10 to about 35 wt. %, about 10 to about 30 wt. %, or about 10 to about 25 wt. %, based on the total weight of the hair lightening composition.

One or more fillers may optionally be included in the hair lightening compositions. Non-limiting examples of fillers include starches, maltodextrins, calcium silicates, perlites, zeolites, polylactic acids, silicas, polyamide powders, polyvinyl-pyrrolidones, dextrose, oligosaccharides, celluloses, diatomite, diatomaceous earth, talc, clays, silicon dioxide, magnesium silicates (i.e. talc powder), clays, and a mixture thereof. In some instances, the one or more fillers comprises one or more alkali metal salts of fatty acids and/or organic base salts of fatty acids, for example, sodium stearate, zinc laurate, magnesium stearate, magnesium myristate, zinc stearate, ammonium stearate, ammonium oleate, ammonium nonanoate, sodium palmitate, potassium stearate, potassium palmitate, sodium myristate, aluminum monostearate, and a mixture thereof.

The total amount of the one or more fillers may vary but is typically about 1 to about 40 wt. %, based on the total weight of the hair lightening composition. In some instances, the total amount of the one or more fillers is about 1 to about 35 wt. %, about 1 to about 30 wt. %, about 1 to about 25 wt. %, about 5 to about 40 wt. %, about 5 to about 35 wt. %, about 5 to about 30 wt. %, about 5 to about 25 wt. %, about 10 to about 40 wt. %, about 10 to about 35 wt. %, about 10 to about 30 wt. %, or about 10 to about 25 wt. %, based on the total weight of the hair lightening composition.

The hair treatment compositions and the hair lightening compositions of the instant disclosure are often anhydrous or essentially anhydrous. The term “essentially anhydrous” means that the composition is either completely free of water or contains no appreciable amount of water, for example, no more than 3% by weight, or no more than 2% by weight, or no more than 1% by weight, based on the total weight of the composition.

A non-limiting example of a hair lightening composition according to the instant disclosure includes: (a) about 0.1 to about 10 wt. % of maleic acid; (b) about 0.01 to about 5 wt. % of one or more aliphatic amino acids selected from the group consisting of glycine, alanine, valine, leucine, isoleucine, and mixtures thereof; (c) about 0.01 to about 10 wt. % of one or more cationic conditioning agents; (d) about 20 to about 80 wt. % of one or more persulfates selected from the group consisting of sodium persulfate, potassium persulfate, ammonium persulfate, and mixtures thereof; (e) about 0.1 to about 10 wt. % of one or more oils; and (f) about 0.1 to about 10 wt. % of one or more thickening agents.

In one embodiment, the instant disclosure relates to essentially anhydrous hair lightening compositions that include: (a) about 0.1 to about 10 wt. %, about 1 to about 8 wt. %, or about 1 to about 5 wt. % of about one or more polycarboxylic acids selected from the group consisting of maleic acid, malonic acid, citric acid, a salt thereof, and a mixture thereof; (b) about 0.01 to about 5 wt. %, about 0.05 to about 4 wt. %, or about 0.1 to about 3 wt. % of one or more aliphatic amino acids selected from the group consisting of glycine, alanine, valine, leucine, isoleucine, a salt thereof, and a mixture thereof; and in particular, glycine and/or a salt thereof; (c) about 0.01 to about 10 wt. %, about 0.05 to about 4 wt. %, or about 0.1 to about 3 wt. % of one or more antioxidant; (d) about 20 to about 80 wt. %, about 30 to about 70, or about 35 to about 65 wt. % of one or more bleaching agents, for example, one or more peroxides, persulfates, perborates, percarbonates, alkali metal bromates, ferricyanides, peroxygenated salts, or a mixture thereof; and in particular one or more persulfates selected from the group consisting of potassium persulfate, sodium persulfate, ammonium persulfate, or a mixture thereof; (e) about 0.1 to about 10 wt. %, about 0.5 to about 8 wt. %, or about 1 to about 5 wt. % of one or more oils, for example, one or more ester oils, silicone oils, fluoro oils, vegetable oils, animal oils, hydrocarbon-based oils, or mixtures thereof; and particularly one or more hydrocarbon-based oils such as mineral oil; (f) about 0.1 to about 10 wt. %, about 0.1 to about 6 wt. %, or about 0.5 to about 5 wt. % of one or more thickening agents, for example, one or more thickening agents selected from the group consisting of acacia, agar, algin, alginic acid, ammonium alginate, amylopectin, calcium alginate, calcium carrageenan, carrageenan, dextrin, gelatin, gellan gum, guar gum, guar hydroxypropyltrimonium chloride, hydroxylpropyl guar hydroxypropyltrimonium chloride, hectorite, hyaluroinic acid, hydrated silica, hydroxypropyl chitosan, hydroxypropyl guar, karaya gum, locust bean gum, natto gum, potassium alginate, potassium carrageenan, propylene glycol alginate, sclerotium gum, sodium carboyxmethyl dextran, sodium carrageenan, tragacanth gum, xanthan gum, acrylates/C10-30 alkylacrylate crosspolymer, and mixtures thereof; and in particular, guar gum; (g) about 0.1 to about 10 wt. %, about 0.1 to about 6 wt. %, or about 0.5 to about 5 wt. % of one or more anionic surfactants, for example, one or more anionic surfactants selected from the group consisting of sodium oleyl succinate, ammonium lauryl sulphosuccinate, sodium lauryl sulfate, sodium lauryl ether sulfate, sodium lauryl ether sulphosuccinate, ammonium lauryl sulfate (ALS), ammonium lauryl ether sulfate, sodium dodecylbenzene sulphonate, triethanolamine dodecylbenzene sulphonate, sodium cocoyl isethionate, sodium lauryl isethionate, lauryl ether carboxylic acid, sodium N-lauryl sarcosinate, and a mixture thereof; and (h) about 1 to about 40 wt. %, about 5 to about 35 wt. %, or about 10 to about 25 wt. % of one or more alkalizing agents, for example one or more alkalizing agents selected from the group consisting of ammonium hydroxide, sodium silicate, sodium metasilicate, monoethanolamine, and a mixture thereof; (i) optionally, about 1 to about 40 wt. %, about 5 to about 35 wt. %, or about 10 to about 25 wt. % of one or more fillers, for example, one or more alkali metal salts of fatty acids and/or organic base salts of fatty acids, such as sodium stearate, zinc laurate, magnesium stearate, magnesium myristate, zinc stearate, ammonium stearate, ammonium oleate, ammonium nonanoate, sodium palmitate, potassium stearate, potassium palmitate, sodium myristate, aluminum monostearate, or a mixture thereof.

The above hair lightening compositions may optionally include one or more cationic conditioning polymers. Alternatively, cationic conditioning polymers may be excluded, i.e., the hair lightening compositions may be free or essentially free of cationic conditioning polymers.

The instant disclosure additionally relates to kits comprising the hair treatment compositions and/or hair lightening compositions. For example, kits can include a hair structuring compositions that are separate from a hair lightening compositions that are separate from an optional aqueous developer composition. Alternatively, kits can include the hair lightening compositions of the instant disclosure (comprising the components of the treatment compositions and bleaching agent(s)) and a separate aqueous developer composition. Typically, the aqueous developer compositions include one or more peroxides, such as hydrogen peroxide. Optionally, the kits may include an additional composition comprising one or more hair conditioning agents and/or one or hair coloring agents.

Finally, the instant disclosure relates to methods for lightening and/or coloring hair; and to methods for protecting and/or improving the appearance of hair, wherein the methods involve applications of the compositions described herein to the hair; allowing the compositions to remain on the hair for a sufficient amount of time for processing; and rinsing the compositions from the hair.

More exhaustive but non-limiting lists of components that are useful in the compositions of the instant disclosure are presented below.

### Antioxidant Compositions

The antioxidant composition according to the disclosure are variously included in the inventive compositions. The antioxidant compositions comprise at least one antioxidant compound. Some representative examples of antioxidant compounds include baicalin (Scutellaria Baicalensis root extract), pine bark extract (Pinus Pinaster bark/bud extract), ferulic acid (a hydroxycinnamic acid) and ellagic acid. Other examples include dihydrochalone, ascorbic acid, zinc PCA, and polydatin. More generally, the antioxidant compounds may be selected from among other phenolic compounds (including polyphenols).

### Phenolic Compounds

Phenolic compounds are a structural class of natural, synthetic, and semisynthetic organic compounds that have one or more phenolic constituents. Phenolic compounds containing multiple phenol groups are known as polyphenols. Polyphenols are normally available in plants and are very helpful to protect plants and also animals from usual health disorders and also the impacts of aging. Polyphenols function as potent free radical scavengers by donating their alcoholic hydrogen or one of their delocalized electrons. The two classes of polyphenols are flavonoids and non-flavonoids.

Flavonoids are a specific group of polyphenols, and are the most plentiful group of polyphenol compounds, making up about two-thirds of the total phenols in consumed feed. Flavonoids are further categorized, according to chemical structure, into chalcones, flavones, flavanones, flavanols, flavonols, dihydroflavonols, isoflavonoids, neoflavonoids, catechins, anthocyanidins, and tannins. Over 4,000 flavonoids have been identified, many of which occur in fruits, vegetables and beverages (tea, coffee, beer, wine and fruit drinks). The flavonoids have been reported to have antiviral, anti-allergic, antiplatelet, anti-inflammatory, antitumor and antioxidant activities. Flavonoids protect lipids and vital cell components from damaging oxidative stress by efficiently scavenging free radicals.

Non-flavonoid polyphenols include lignans, aurones, stilbenoids, curcuminoids and other phenylpropanoids. Many of them are also well-known antioxidants like resveratrol, curcumin, and pinoresinol.

Other phenolic compounds, in addition to polyphenols, include alkylphenols, betacyanins, capsacinoids, hydroxybenzoketones, methoxyphenols, naphthoquinones, and phenolic terpenes. Some popular examples are ferulic acid, hydroxytyrosol, cinnamic acid, caffeic acid, and p-coumaric acid.

### Selected Antioxidant Compounds

In accordance with some particular examples according to the disclosure, antioxidant compounds include one or more of baicalin (Scutellaria Baicalensis root extract), pine bark extract (Pinus Pinaster bark/bud extract), ferulic acid (a hydroxycinnamic acid) and ellagic acid.

Scutellaria Baicalensis root extract includes the compound baicalin, which has also been identified as a component of Chinese medicinal herb Huang-chin, is a flavone, a type of flavonoid. It is a potent antioxidant that demonstrates potent effects against oxidative stress diseases, inflammation, allergy, cancer, bacterial infections, etc.

Pine bark extract (Pinus Pinaster bark/bud extract) contains a variety of compounds, including oligomeric proanthocyanidin compounds, and is generally associated with benefits that range from cholesterol control to cardiovascular, other circulatory and brain benefits.

Ferulic acid is a hydroxycinnamic acid that can be broadly found in giant fennel, the seeds of coffee, apple, artichoke, peanut, and oranges, as well as in both seeds and cell walls of commelinid plants (such as rice, wheat, oats, and pineapple). Like many natural phenols, it is a strong antioxidant that is very reactive toward free radicals and reduces oxidative stress. Many studies suggest that ferulic acid may have antitumor activity.

Ellagic acid is a small phytochemical that occurs naturally in red fruits and berries and in some nuts, including pomegranate, strawberries, blackberries, raspberries, cranberries, walnuts and pecans. Ellagic acid can also be generated by breaking down ellagitannins that can be obtained from some plants. Ellagic acid has been shown to have anticancer activity among other benefits.

In accordance with the disclosure, the antioxidant compositions comprise an antioxidant up to 100%, and may comprise a diluent or carrier or other component, such as for example, sodium silicate. In some embodiments, the antioxidant compositions include an antioxidant active that is present at a concentration, by weight, of about 50%. Thus, in accordance with the inventive compositions, a composition comprising (a) an antioxidant composition comprising at least one antioxidant compound, and a color altering system that includes (b) a bleach, and (c) a developer, or (c) a developer and (d) a colorant which can be selected from oxidative dye precursors, wherein, according to methods of use of the composition, (i) (a) and one or more of (b) and (c), or (ii) (a) and one or more of (c)and (d) are combined or combinable, the antioxidant composition may comprise an antioxidant active that is present up to 100%, or may be present in an amount that is less than 100%, such as for example, 50%.

Thus, antioxidants are used in the compositions as raw materials that include from about 0.1% or less, to about 100% of at least one antioxidant active, and are in some examples provided in a mixture together with a component, such as, but not limited to, sodium silicate, where the at least one antioxidant active is present in the mixture from about 0.1% to about 99%, by weight, based on the total weight of the raw material.

In accordance with the various embodiments as set forth herein below, the compositions, kits and methods of the invention include with the at least one antioxidant, one or more components of a color altering system selected from the group consisting of: a bleach, a developer and a colorant selected from oxidative dye precursors and direct dyes. Embodiments that are useful for color lightening or lifting comprise a bleach as an oxidizer and a developer as an oxidizing agent. Embodiments that are useful for color changing or depositing comprise a colorant and a developer. Thus, in the various embodiments, compositions and mixtures include at least one antioxidant, one or the other of a bleach and a colorant, and a developer.

In a representative example, as further described in the examples herein below, a composition comprising (a) an antioxidant composition comprising at least one antioxidant compound, and a color altering system that includes (b) one of a bleach and a colorant, and (c) a developer, wherein, according to methods of use of the composition, (a) and one or more of (b) and (c), are combined or combinable includes 15 g of bleach and 0.2 g of a composition that comprises 50% baicalin active. Thus, the amount of baicalin active present is 0.1 g. Expressed as a weight percent of the combination of bleach and baicalin, the baicalin is present in the compositions at about 0.7% by weight, based on the weight of the composition comprising the baicalin antioxidant and the bleach. For purposes of this example, the amount of developer is not considered in the calculation of the total weight of the composition.

Reference is made to the examples herein below with respect to the absolute weight amounts of each of the other exemplified antioxidant compositions, wherein for each of them, the weight percent thereof may be expressed as the percentage by weight of the combination of the antioxidant and the bleach.

Thus, in accordance with the disclosure, the amount of baicalin active that is present in the inventive a composition comprising (a) an antioxidant composition comprising at least one antioxidant compound, and a color altering system that includes (b) one of a bleach and a colorant, and (c) a developer, wherein, according to methods of use of the composition, (a) and one or more of (b) and (c) are combined or combinable can range from at least about 0.1%, and up to about 5%, based on the weight of the total composition comprising the antioxidant and the bleach. In some particular embodiments, the amount of baicalin can range from about 0.2% to about 4%; or from about 0.3% to about 3.5%; or from about 0.7% to about 2.3%, based on the total weight of the composition comprising the antioxidant and the bleach. In accordance with some embodiments, the compositions include not more than 2.5% baicalin. In accordance with other embodiments, the compositions include not more than 2.3%, or not more than 2% or not more than 1.5%, or not more than 1%, or not more than 0.7%, or not more than 0.5% baicalin.

Thus, in accordance with the disclosure, the amount of pine bark extract active that is present in the inventive a composition comprising (a) one or more polycarboxylic acid and amino acid in combination with an antioxidant composition comprising at least one antioxidant compound, and a color altering system that includes (b) one of a bleach and a colorant, and (c) a developer, wherein, according to methods of use of the composition, (a) and one or more of (b) and (c) are combined or combinable can range from at least about 0.1%, and up to about 5%, based on the weight of the total composition comprising the antioxidant and the bleach. In some particular embodiments, the amount of pine bark extract can range from about 0.2% to about 4%; or from about 0.3% to about 3.5%; or from about 0.7% to about 2.3%, based on the total weight of the composition comprising the antioxidant and the bleach. In accordance with some embodiments, the compositions include not more than 2.5% pine bark extract. In accordance with other embodiments, the compositions include not more than 2.3%, or not more than 2% or not more than 1.5%, or not more than 1%, or not more than 0.7%, or not more than 0.5% pine bark extract.

Thus, in accordance with the disclosure, the amount of ferulic acid active that is present in the inventive a composition comprising (a) an antioxidant composition comprising at least one antioxidant compound, and a color altering system that includes (b) one of a bleach and a colorant, and (c) a developer, wherein, according to methods of use of the composition, (a) and one or more of (b) and (c), are combined or combinable can range from at least about 0.1%, and up to about 3%, based on the weight of the total composition comprising the antioxidant and the bleach. In some particular embodiments, the amount of ferulic acid can range from about 0.2% to about 2%; or from about 0.3% to about 1.5%; or from about 0.7% to about 1.2%, based on the total weight of the composition comprising the antioxidant and the bleach. In accordance with some embodiments, the compositions include not more than 2% ferulic acid. In accordance with other embodiments, the compositions include not more than 1.8%, or not more than 1.6% or not more than 1.4%, or not more than 1.2%, or not more than 0.8%, or not more than .5% ferulic acid.

Thus, in accordance with the disclosure, the amount of ellagic acid active that is present in the inventive a composition comprising (a) an antioxidant composition comprising at least one antioxidant compound, and a color altering system that includes (b) one of a bleach and a colorant, and (c) a developer, wherein, according to methods of use of the composition, (a) and one or more of (b) and (c), are combined or combinable can range from at least about 0.1%, and up to about 3%, based on the weight of the total composition comprising the antioxidant and the bleach. In some particular embodiments, the amount of ellagic acid can range from about 0.2% to about 2.5%; or from about 0.6% to about 2.0%; or from about 0.9% to about 1.8%, based on the total weight of the composition comprising the antioxidant and the bleach. In accordance with some embodiments, the compositions include not more than 2.5% ellagic acid. In accordance with other embodiments, the compositions include not more than 2.3%, or not more than 2% or not more than 1.5%, or not more than 1.2%, or not more than 0.9%, or not more than 0.5% ellagic acid.

As described herein, an antioxidant may be provided in a raw material form that may include one or more additional components, wherein the amount of an antioxidant active in such raw material composition may range from 0.1% to 99% active. Thus, in various embodiments, any one or more of antioxidant components may be present in any composition according to the disclosure in a weight percent amount as described herein above, that is determined as the product of the percentage purity (based upon weight, unless otherwise stated) of the antioxidant in the raw material composition and the percentage, by weight, of the raw material composition used in the inventive composition. Thus, one or more antioxidant actives may be present in an antioxidant raw material composition in an amount that ranges from 0.01 to about 99%, or from about 0.01, 0.05, 0.1, 0.2, 0.3, 04, 0.5, 0.6, 0.7, 0.8, 0.9, 1, 2, 3, 4, 5, 6, 7, 8, 9, 10, 15, 20, 25, 30, 35, 40, 45, 50, 55, 60, 65, 70, 75, 80, 85, 90, 95 to about 99 percent by weight, including increments and ranges therein and there between. And an inventive composition comprising at least one antioxidant active, and one or more of a bleach, a developer, a colorant, a carrier, and an additive or auxiliary, will comprise antioxidant active from about 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2, 3, 4, to about 5 percent by weight, including increments and ranges therein and there between. In one example, an inventive composition includes 0.4% by weight of an antioxidant raw material composition that comprises 50% antioxidant active; thus, the inventive composition comprises 0.2% antioxidant active.

### Additives

### Optional Additional Antioxidants

The compositions can also contain one or more additional antioxidants that is/are different from the flavonoid(s) used in the composition and ferulic acid.

Additional antioxidants can be any antioxidant suitable for use in cosmetic formulations. Suitable antioxidants include, but are not limited to, resveratrol, tannic acid, polyphenols, amino acids and derivatives thereof, imidazoles, peptides such as carnosine and derivatives, carotenoids, carotenes (such as α-carotene, β-carotene, and lycopene), α-hydroxy acids (such as citric acid, lactic acid, or malic acid), tocopherols and derivatives (such as Vitamin E), vitamin A, ascorbic acid, co-enzyme Q10, bioflavonoids, glutathione, plant extracts (such as rosemary extract, olive leaf extracts), and green tea extracts.

The amount of additional antioxidants present in the compositions can range from about 0.01% to about 20%; about 0.1% to about 20%; or about 0.01% to about 10%, based on the total weight of the composition.

### Optional Hydrotropes

Hydrotropes or glycols may be used in some embodiments to increase solubility of the components of the compositions if solubility of any of the components in water is low. A hydrotrope solution is prepared by completely dissolving one or more hydrotropic agents into water. The component(s) are then added in and mixed using stirring bar or any other mixer. Solubilization of the components occurs within minutes, and mixing continued until clear stable solution is obtained, usually within one hour of mixing. No heat is necessary by following this procedure to dissolve phenolic compounds. Everything is prepared at room temperature to keep the stability of phenolic compounds. This is extremely useful to protect the activity of certain compounds and also makes the process much easier.

Hydrotropes (or hydrotropic agents) are a diverse class of water-soluble compounds that are characterized by an amphiphilic molecular structure and ability to dramatically increase the solubility of poorly soluble organic molecules in water. The at least one hydrotrope is present in the composition in amounts effective to increase the solubility of the phenolic compound in water. At least one hydrotrope refers to one or a combination of two or more hydrotropes. One or a combination of two or more hydrotropes can be used to improve the solubility of phenolic compounds in water. The amount of hydrotrope will vary depending on the hydrotrope and the type and amount of phenolic compound. Increasing the water solubility of the phenolic compound(s) refers to increasing the solubility of the phenolic compound(s) in water in comparison with solubility of the phenolic compound(s) in water in the absence of the hydrotrope or hydrotropes.

An advantage of using hydrotropes is, once a stable solution is obtained, further dilution doesn't influence the stability of the solution. This is very different from organic solvents that are commonly used to increase the water solubility of phenolic compounds, such as polyphenols. Typically, an aqueous dilution of organic solvents with pre-dissolved phenolic compound(s), such as a polyphenol, results in crystallization or precipitation.

Most hydrotropes have aromatic structure with an ionic moiety, while some of them are linear alkyl chains, as listed in the table below. Although hydrotropes noticeably resemble surfactants and have the ability to reduce surface tension, their small hydrophobic units and relatively shorter alkyl chain distinguish them as a separate class of amphiphiles. Consequently, their hydrophobicity is not sufficient enough to create well organized self-associated structures, such as micelles, even with a high concentration.

Common hydrotropic molecules include: sodium 1,3-benzenedisulfonate, sodium benzoate, sodium 4-pyridinecarboxylate, sodium salicylate, sodium benzene sulfonate, caffeine, sodium p-toluene sulfonate, sodium butyl monoglycolsulfate, 4-aminobenzoic acid HCl, sodium cumene sulfonate, N,N-diethylnicotinamide, N-picolylnicotinamide, N-allylnicotinamide, 2-methacryloyloxyethyl phosphorylcholine, resorcinol, butylurea, pyrogallol, N-picolylacetamide 3.5, procaine HCl, proline HCl, nicotinamide, pyridine, 3-picolylamine, sodium ibuprofen, sodium xylenesulfonate, ethyl carbamate, pyridoxal hydrochloride, sodium benzoate, 2-pyrrolidone, ethylurea, N,N-dimethylacetamide, N-methylacetamide, and isoniazid. Hydrotropes can be found in Lee J. et al., “Hydrotropic Solubilization of Paclitaxel: Analysis of Chemical Structures for Hydrotropic Property”, Pharmaceutical Research, Vol. 20, No. 7, 2003; and Lee S. et al., “Hydrotropic Polymers: Synthesis and Characterization of Polymers Containing Picolylnicotinamide Moieties”, Macromolecules, 36, 2248-2255, 2003.

Cosmetically acceptable hydrotropes refers to hydrotropes that can be used in cosmetic compositions. While hydrotropes represent a broad class of molecules used in various fields, cosmetic applications will be limited due to safety and tolerance restrictions. Suitable hydrotropes for use in cosmetics include, but are not limited to, nicotinamide (vitamin B3), caffeine, sodium PCA (sodium salt of pyrrolidone carbonic acid), sodium salicylate, urea, and hydroxyethyl urea. The suitability of a hydrotrope for use in cosmetic compositions can be determined using tests known in the art for determining effects on skin, and toxicity to humans.

The amount of hydrotropes present in the compositions can range from about 0.1% to about 20%; about 0.1% to about 10%; or about 1% to about 50%, based on the total weight of the composition.

### Water

The antioxidant containing compositions can contain from about 1 to about 99.9% by weight of water, with respect to the total weight of the composition. The amount of water in the composition can range from about 1 to 99.5%; about 1 to 60%; or about 1 to 50%, based on the total weight of the composition.

pH

The pH of the compositions is not limited but is generally between 2 and 12, or between 3 and 9. The pH can be adjusted to the desired value by addition of a base (organic or inorganic) to the composition, for example ammonia or a primary, secondary or tertiary (poly)amine, such as monoethanolamine, diethanolamine, triethanolamine, isopropanolamine or 1,3-propanediamine, or alternatively by addition of an inorganic or organic acid, advantageously a carboxylic acid, such as, for example, citric acid.

### Polycarboxylic Acids

The polycarboxylic acid may be chosen especially from linear, branched and/or cyclic, saturated or unsaturated, or even aromatic, polycarboxylic acids, containing 2 to 50, especially 2 to 40 carbon atoms, in particular 3 to 36, or even 3 to 18, 4 to 12 carbon atoms, or even 5 to 10 carbon atoms; the acid comprising at least two carboxylic groups COOH, or from 2 to 4 COOH groups; and possibly comprising 1 to 10 or 1 to 6 identical or different heteroatoms, chosen from O, N and S; and/or possibly comprising at least one perfluoro radical chosen from —CF₂— (divalent) or —CF₃.

In some cases, the said polycarboxylic acid is saturated, linear and aliphatic and contains 2 to 36 carbon atoms, especially 3 to 18 carbon atoms or even 4 to 12 carbon atoms; or alternatively is aromatic and contains 8 to 12 carbon atoms. In some cases, it may comprise 2 to 4 COOH groups.

The cyclic anhydride of a polycarboxylic acid may especially correspond to one of the following formulae:

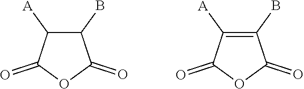

in which the groups A and B are, independently of each other: a hydrogen atom, a saturated or unsaturated, linear, branched and/or cyclic aliphatic, or alternatively aromatic, carbon-based radical; containing 1 to 16 carbon atoms, especially 2 to 10 carbon atoms or even 4 to 8 carbon atoms, especially methyl or ethyl, or alternatively A and B taken together form a saturated or unsaturated, or even aromatic, ring comprising in total 5 to 14, especially 5 to 10 or even 6 to 7 carbon atoms. In some cases, A and B represent a hydrogen atom or together form an aromatic ring containing in total 6 to 10 carbon atoms.

Among the polycarboxylic acids or anhydrides thereof that may be used, mention may be made, alone or as a mixture, of: dicarboxylic acids such as decanedioic acid, dodecanedioic acid, cyclopropanedicarboxylic acid, cyclohexanedicarboxylic acid, cyclobutanedicarboxylic acid, naphthalene-1,4-dicarboxylic acid, naphthalene-2,3-dicarboxylic acid, naphthalene-2,6-dicarboxylic acid, suberic acid, oxalic acid, malonic acid, succinic acid, phthalic acid, terephthalic acid, isophthalic acid, tetrahydrophthalic acid, hexahydrophthalic acid, pimelic acid, sebacic acid, azelaic acid, glutaric acid, adipic acid, fumaric acid, maleic acid, itaconic acid and fatty acid dimers (especially of C₃₆); tricarboxylic acids such as cyclohexanetricarboxylic acid, trimellitic acid, 1,2,3-benzenetricarboxylic acid and 1,3,5-benzenetricarboxylic acid, tetracarboxylic acids such as butanetetracarboxylic acid and pyromellitic acid, cyclic anhydrides of these acids and especially phthalic anhydride, trimellitic anhydride, maleic anhydride and succinic anhydride.

Mention may also be made of polycarboxylic acids chosen, alone or as a mixture, from:

(i) polycarboxylic acids containing a saturated or unsaturated, linear or branched chain comprising at least one heteroatom chosen from O, N and/or S, especially 1 to 10 identical or different heteroatoms, and/or comprising at least one perfluoro radical —CF₂— or —CF₃ and moreover containing at least 2 carboxylic groups COOH and especially 2 to 4 COOH groups; and/or a cyclic anhydride of such a polycarboxylic acid; and/or

(ii) saturated or unsaturated, or even aromatic, heterocyclic polycarboxylic acids, comprising at least one heteroatom chosen from O, N and/or S, especially 1 to 10, or even 1 to 4, identical or different heteroatoms, and at least 2 carboxylic groups COOH and especially 2 to 4 COOH groups; and/or a cyclic anhydride of such a polycarboxylic acid; and/or

(iii) sugar-based polycarboxylic acids, which may be obtained especially by oxidation of an aldose, and comprising at least 2 carboxylic groups COOH and especially 2 or 3 COOH groups; and/or a cyclic anhydride of such a polycarboxylic acid; and/or

(iv) itaconic anhydride and 1,4,5,8-naphthalenetetracarboxylic acid 1,4-monoanhydride; and/or

(v) polycarboxylic (including heterocyclic) amino acids, i.e. polycarboxylic acids containing a saturated or unsaturated, linear, branched and/or cyclic chain, optionally comprising at least one heteroatom chosen from O, N and/or S, especially 1 to 10 identical or different heteroatoms, and/or optionally comprising at least one perfluoro radical —CF₂— or —CF₃; and also comprising at least one primary, secondary or tertiary amine function (especially NR¹R² with R¹ and R², independently of each other, chosen from H and C₁-C₁₂ alkyl), especially 1 to 3 identical or different amine functions, and moreover containing at least 2 carboxylic groups COOH, especially 2 to 4 COOH groups; and/or a cyclic anhydride of such a polycarboxylic acid.

Mention may be made most particularly, alone or as a mixture, of the following dicarboxylic acids: (i) 2,2′-[1,5-pentanediylbis(thio)]bis-acetic acid 6,6′-[(1,2-dioxo-1,2-ethanediyl)diimino]bis-hexanoic acid 2,2′-sulfinylbis-acetic acid 4,13-dioxo-3,5,12,14-tetraazahexadecanedioic acid poly(ethylene glycol)disuccinate, especially of mass 250-600 poly(ethylene glycol)bis(carboxymethyl) ether, especially of mass 250-600 poly[oxy(1,2-dicarboxy-1,2-ethanediyl)], especially of DP<1 0 8-[(carboxymethyl)amino]-8-oxooctanoic acid 2,2′-[methylenebis(sulfonyl)]bis-acetic acid 4,4′-(1,6-hexanediyldiimino)bis[4-oxobutanoic acid] 4,9-dioxo-3,5,8,10-tetraazadodecanedioic acid 4-[(1-carboxyethyl)amino]-4-oxobutanoic acid 6-[(3-carboxy-1-oxopropyl)amino]hexanoic acid N,N′-(1,6-dioxo-1,6-hexanediyl)bis-glycine N,N′-(1,6-dioxo-1,6-hexanediyl)bis-phenylalanine N,N′-(1,3-dioxo-1,3-propanediyl)bis-glycine 4,4′-[(1,4-dioxo-1,4-butanediyl)diimino]bis-butanoic acid 4,4′-[(1,6-dioxo-1,6-hexanediyl)diimino]bis-butanoic acid 6,6′-[1,6-hexanediylbis(iminocarbonylimino)]bis-hexanoic acid N-benzoyl-S-(carboxymethyl)cysteine N,N′-(2,2,3,3-tetrafluoro-1,4-dioxo-1,4-butanediyl)bis-glycine N,N′-(2,2,3,3-tetrafluoro-1,4-dioxo-1,4-butanediyl)bis-alanine 4,4′-[(2,2,3,3-tetrafluoro-1,4]-dioxo-1,4-butanoic acid N,N′-(1,5-dioxo-1,5-pentanediyl)bis-glycine N,N′-(1,9-dioxo-1,9-nonanediyl)bis-glycine N,N′-(1,10-dioxo-1,10-decanediyl)bis[N-methyl]glycine bis(3-carboxypropyl)ester of propanedioic acid 7,16-dioxo-6,8,15,17-tetraazadocosanedioic acid N-benzoyl-N-(2-carboxyethyl)glycine 2-[(2-carboxymethyl)amino]-2-oxoethypenzenepropanoic acid [2-4(2-carboxyethyl)amino]-2-oxoethypenzenepropanoic acid (ii) 4,7,9,12-tetraoxapentadecanedioic acid 2,3-pyridinedicarboxylic acid 4-pyranone-2,6-dicarboxylic acid 2,5-pyrazinedicarboxylic acid 2,5-pyridinedicarboxylic acid 2,3-benzofurandicarboxylic acid 7-oxabicyclo[2.2.1]heptane-2,3-dicarboxylic acid 3,4-pyridinedicarboxylic acid 2,4-pyridinedicarboxylic acid 3,5-pyridinedicarboxylic acid 2,6-pyridinedicarboxylic acid 1H-imidazole-4,5-dicarboxylic acid 2,3-quinolinedicarboxylic acid 6,6,7,7-tetrafluoro-3-oxabicyclo[3.2.0]heptane-2,4-dicarboxylic acid 2,6-pyrazinedicarboxylic acid 2,6-dimethyl-3,5-pyridinedicarboxylic acid 1-phenyl-1H-pyrazole-3,4-dicarboxylic acid 2,5-furandicarboxylic acid 3,4-furandicarboxylic acid 1,2,5-thiadiazole-3,4-dicarboxylic acid 1,4-dihydro-1,2,4,5-tetrazine-3,6-dicarboxylic acid 2,3-furandicarboxylic acid 3,4-thiophenedicarboxylic acid 1H-1,2,3-triazole-4,5-dicarboxylic acid 2-methylimidazole-4,5-dicarboxylic acid 2,4-quinolinedicarboxylic acid naphtho[2,1-b]furan-1,2-dicarboxylic acid 3,4-quinolinedicarboxylic acid 7-oxabicyclo[2.2.1]hept-5-ene-2,3-dicarboxylic acid 2,3-quinoxalinedicarboxylic acid 1,4-piperazinedicarboxylic acid 2,5-dimethyl-3,4-furandicarboxylic acid tetrahydro-2,5-thiophenedicarboxylic acid 4-phenyl-3,5-pyridinedicarboxylic acid thieno[3,2-b]thiophene-2,5-dicarboxylic acid 3-methyl-2,4-thiophenedicarboxylic acid naphthostyril-5,6-dicarboxylic acid 3-phenyl-2,4-quinolinedicarboxylic acid 3,4-dimethyl-2,5-dicarboxythiophene 3,4-diphenyl-2,5-thiophenedicarboxylic acid 2,5-diphenyl-3,4-furandicarboxylic acid 7-oxo-7H-benzimidazo[2,1-a]benz[de]isoquinoline-3,4-dicarboxylic acid 2,3-dihydro-1,3-dioxo-1H-benz[de]isoquinoline-6,7-dicarboxylic acid 3,4-bis(phenylmethoxy)-2,5-furandicarboxylic acid 4,4′-bibenzoic acid-2,2′-sulfone 2,7-diphenyl-m-anthrazoline-4,5-dicarboxylic acid 2,4-pyrimidinedicarboxylic acid 2-phenyl-4,5-thiazoledicarboxylic acid 6-phenyl-2,3-pyridinedicarboxylic acid 5,6-dimethyl-2,3-pyrazinedicarboxylic acid 3,7-dibenzothiophenedicarboxylic acid 9-oxo-9H-xanthene-1,7-dicarboxylic acid 2-(1,1-dimethylethyl)-H-imidazole-4,5-dicarboxylic acid 6,7-quinolinedicarboxylic acid 6-methyl-2,3-pyridinedicarboxylic acid 4,5-pyrimidinedicarboxylic acid 2-methyl-3,4-furandicarboxylic acid 1,2-indolizinedicarboxylic acid 2,8-dibenzothiophenedicarboxylic acid 3,6-pyridazinedicarboxylic acid 1,10-phenanthroline-2,9-dicarboxylic acid 1,4,5,6-tetrahydro-5,6-dioxo-2,3-pyrazinedicarboxylic acid 3,4-dimethoxy-2 ,5-f urandicarboxylic acid 2-ethyl-4,5-imidazoledicarboxylic acid 2-propyl-1 H-imidazole-4,5-dicarboxylic acid 4-phenyl-2,5-pyridinedicarboxylic acid 4,5-pyridazinedicarboxylic acid 1,4,5,8-tetrahydro-1,4:5,8-diepoxynaphthalene-4a,8a-dicarboxylic acid 5,5-dioxide-2,8-dibenzothiophenedicarboxylic acid pyrazolo[1,5-a]pyridine-2,3-dicarboxylic acid 2,3-dihydro-1H-pyrrolizine-1,7-dicarboxylic acid 6-methyl-2,4,5-pyridinetricarboxylic acid pyrrolo[2,1,5-cd]indolizine-5,6-dicarboxylic acid 3,4-bis(2,2,3,3,4,4,4-heptafluorobutyl)-1H-pyrrole-2,5-dicarboxylic acid 6,7,9,10,17,18,20,21-octahydrodibenzo[b,k]-[1,4,7,10,13,16]hexaoxacyclooc- tadecin-2,14-dicarboxylic acid 6,7,9,10,17,18,20,21-octahydrodibenzo[b,k]-[1,4,7,10,13,16]hexaoxacyclooctadecin-2,13-dicarboxylic acid 2-methyl-3,4-quinolinedicarboxylic acid 4,7-quinolinedicarboxylic acid 3,5-isoxazoledicarboxylic acid 2-(trifluoromethyl)-3,4-furandicarboxylic acid 5-(trifluoromethyl)-2,4-furandicarboxylic acid 6-methyl-2,4-quinolinedicarboxylic acid 5-oxo-1,2-pyrrolidinedicarboxylic acid 5-ethyl-2,3-pyridinedicarboxylic acid 1,2-dihydro-2-oxo-3,4-quinolinedicarboxylic acid 4,6-phenoxathiindicarboxylic acid 10,10-dioxide 1,9-phenoxathiindicarboxylic acid 3,4-dihydro-2H-1,4-thiazine-3,5-dicarboxylic acid 2,7-di(tert-butyl)-9,9-dimethyl-4,5-xanthenedicarboxylic acid 6-methyl-2,3-quinoxalinedicarboxylic acid 3,7-quinolinedicarboxylic acid 2,5-quinolinedicarboxylic acid 2-methyl-6-phenyl-3,4-pyridinedicarboxylic acid 3,4-dimethylthieno[2,3-b]thiophene-2,5-dicarboxylic acid 3,4-dimethoxythiophene-2,5-dicarboxylic acid 5-methyl-3,4-isoxazoledicarboxylic acid 2,6-bis(aminocarbonyl)-3,5-pyridinedicarboxylic acid 3,5-bis(aminocarbonyl)-2,6-pyrazinedicarboxylic acid 2,3-pyridinedicarboxylic acid 6-(1,1-dimethylethyl)-2-ethyl-3,4-pyridinedicarboxylic acid 3-methyl-5-phenyl-2,4-thiophenedicarboxylic acid 1,2-dihydro-2-oxo-6-phenyl-3,5-pyridinedicarboxylic acid 8-methyl-2,4-quinolinedicarboxylic acid 4-ethyl-2,6-dimethyl-3,5-pyridinedicarboxylic acid 5-(phenoxymethyl)-2,4-furandicarboxylic acid 5-(acetylamino)-3-methyl-2,4-thiophenedicarboxylic acid 2-(4-heptylphenyl)-4,8-quinolinedicarboxylic acid 2,8-bis(4-heptylphenyl)pyrido[3,2-g]quinoline-4,6-dicarboxylic acid 1,2,3,4,6,7,8,9-octahydro-2,8-dioxopyrido[3,2]-quinoline-3,7-dicarboxylic acid 2,8-dimethylpyrido[3,2-g]quinoline-3,7-dicarboxylic acid 5,6-quinolinedicarboxylic acid 6-ethyl-2-methylcinchomeronic acid 2-methyl-6-propylcinchomeronic acid 6-isopropyl-2-methylcinchomeronic acid 6-tert-butyl-2-methylcinchomeronic acid 1,4-dimethyl-7-oxabicyclo[2.2.1]heptane-2,3-dicarboxylic acid 1,2-dihydro-2-oxo-3,8-quinolinedicarboxylic acid 1,2-dihydro-2-oxo-3,6-quinolinedicarboxylic acid 1,2-dihydro-2-oxo-3,7-quinolinedicarboxylic acid 3,7-dimethyl-2,8-diphenylpyrido[3,2-g]quinoline-4,6-dicarboxylic acid 8-methyl-2,3-quinolinedicarboxylic acid 3-[[(1,1-dimethylethyl)amino]sulfonyl]-2,5-thiophenedicarboxylic acid 4-(acetylamino)-2,3-thiophenedicarboxylic acid 2,5-pyridinedicarboxylic acid 2,6-pyridinedicarboxylic acid 2,4-thiophenedicarboxylic acid 2,5-thiophenedicarboxylic acid 1,4-pyran-2,6-dicarboxylic acid (iii) ribaric acid glucaric acid xylaric acid arabinaric acid mannaric acid idaric acid altraric acid L-glucaric acid L-arabinaric acid allaric acid galactaric acid meso-tartaric acid D-glucaric acid L-idaric acid hexaric acid 2,3-dihydroxybutanedioic acid D-tartaric acid D,L-tartaric acid D-glucaric acid tartaric acid tetrahydroxysuccinic acid 2-carboxy-2,3-dideoxy-D-manno-2-octulopyranosonic acid methyl-3-deoxy-D-arabino-2-heptulopyranosaric acid D-lyxo-2-heptulopyranosaric acid 2,6-anhydro-L-glycero-L-galactoheptaric acid (iv) 1,4,5,8-naphthalenetetracarboxylic acid 1,4-monoanhydride itaconic anhydride (v) 1,4-dihydro-4-oxo-2,6-pyridinedicarboxylic acid 2,6-piperidinedicarboxylic acid 1H-pyrrole-3,4-dicarboxylic acid 4-amino-2,6-dicarboxylic acid 1-methyl-1H-pyrazole-3,4-dicarboxylic acid 2,3-piperidinedicarboxylic acid 1-methyl-1H-imidazole-4,5-dicarboxylic acid 2,4-thiazolidinedicarboxylic acid 1-(phenylmethyl)-1H-imidazole-4,5-dicarboxylic acid 5-amino-6-oxo-2,3-piperidinedicarboxylic acid 5-amino-6-oxo-2,4-piperidinedicarboxylic acid 5-amino-6-oxo-2,3-piperidinedicarboxylic acid 5-amino-6-oxo[2S-(2.alpha.,4.beta.,5.alpha.)]-2,4-piperidinedicarboxylic acid (2S,4R)-2,4-pyrrolidinedicarboxylic acid (2S-cis)-2,4-pyrrolidinedicarboxylic acid 2-amino-1 H-imidazole-4,5-dicarboxylic acid 2,5-pyrrolidinedicarboxylic acid 4-amino-3,5-isothiazoledicarboxylic acid 1-methyl-1H-pyrazole-3,5-dicarboxylic acid 7-(diethylamino)-2-oxo-2H-1-benzopyran-3,4-dicarboxylic acid 3,4-diethyl-1H-pyrrole-2,5-dicarboxylic acid 1-phenyl-1H-pyrrole-3,4-dicarboxylic acid cis-2,3-piperazinedicarboxylic acid 2,3-piperazinedicarboxylic acid 2,5-piperazinedicarboxylic acid 2,6-piperazinedicarboxylic acid 2-amino-3,5-pyridinedicarboxylic acid 2-methylpyrrole-3,4-dicarboxylic acid 4-(methylamino)-2,6-pyridinedicarboxylic acid 2-amino-6-methyl-3,4-pyridinedicarboxylic acid 5-amino-2-methyl-3,4-pyridinedicarboxylic acid 2-amino-6-methyl-3,5-pyridinedicarboxylic acid 2,5-dimethyl pyrrole-3,4-dicarboxylic acid 2,5-dimethylpyrrole-3,4-dicarboxylic acid 2-amino-6-hydroxy-3,5-pyridinedicarboxylic acid 2,4-pyrrolidinedicarboxylic acid 1H-indole-2,4-dicarboxylic acid 1H-indole-2,6-dicarboxylic acid 1H-indole-2,5-dicarboxylic acid 5-phenyl-2,4-pyrrolidinedicarboxylic acid 5-methyl-2,4-pyrrolidinedicarboxylic acid trans-2,4-azetidinedicarboxylic acid cis-2,4-azetidinedicarboxylic acid 3,5-piperidinedicarboxylic acid 2,3-pyrrolidinedicarboxylic acid 2,3-azetidinedicarboxylic acid 3,4-pyrrolidinedicarboxylic acid 2,3-dihydro-6H-1,4-dioxino[2,3-c]pyrrole-5,7-dicarboxylic acid 1H-imidazole-2,4-dicarboxylic acid 1-butyl-1H-pyrrole-2,3-dicarboxylic acid 3-amino-1-oxide-2,4-pyridinedicarboxylic acid 2,3-dihydro-5-phenyl-1H-pyrrolizine-6,7-dicarboxylic acid 3a,4,5,9b-tetrahydro-3H-cyclopenta[c]quinoline-4,6-dicarboxylic acid 3a,4,5,9b-tetrahydro-3H-cyclopenta[c]quinoline-4,8-dicarboxylic acid 2,3-dihydro-1H-imidazole-4,5-dicarboxylic acid 5-amino-6-methyllutidinic acid 1H-indole-3,7-dicarboxylic acid 3,3-dimethyl-2,6-piperidinedicarboxylic acid 1-butyl-2,5-pyrrolidinedicarboxylic acid 1H-indole-4,6-dicarboxylic acid 1-(phenylmethyl)-3,4-pyrrolidinedicarboxylic acid 3-(carboxymethyl)-1H-indole-2,6-dicarboxylic acid 3,4-bis(2,2,2-trifluoroethyl)-1H-pyrrole-2,5-dicarboxylic acid 9-hexyl-9H-carbazole-3,6-dicarboxylic acid 3-methyl-5-(1-piperazinylsulfonyl)-2,4-thiophenedicarboxylic acid 2,3,4,9-tetrahydro-1H-carbazole-5,7-dicarboxylic acid 2,3-dimethyl-1H-indole-4,6-dicarboxylic acid 7-amino-1,4-dihydro-4-oxo-3,6-quinolinedicarboxylic acid 5-amino-3-methyl-2,4-thiophenedicarboxylic acid (m-tolylimino)diacetic acid (o-tolylimino)diacetic acid D-cystathionine phenethyliminodiacetic acid 2-benzyl-2,2′-iminodiacetic acid L-.alpha.-glutamyl-L-alanyl-L-alanine N,N′-dibenzylethylenediaminediacetic acid N-L-.gamma.-glutamyl-D-alanine glycyl-L-glutamylglycine N-(carboxymethyl)-N-(tetrahydro-1,1-dioxido-3-thienyl)glycine N-(2-carboxyethyl)-N-phenyl-beta-alanine N-(carboxymethyl)-N-octylglycine N-(tert-butoxycarbonyl)iminodiacetic acid N-(carboxymethyl)-L-alanine N-(6-aminohexyl)-N-(carboxymethyl)glycine N-(carboxymethyl)-N-tetradecylglycine N-(1-carboxyethyl)-D-alanine N-(carboxymethyl)-D-alanine decyliminodiacetic acid 3,3′-(dimethylhydrazono)bis-propanoic acid N-(carboxymethyl)-N-[2-(2,6-dioxo-4-morpholinyl)ethyl]glycine N-alpha-aspartylglycine N-beta-aspartylglycine N-L-alpha-aspartyl-beta-alanine 3,4-xylylamino-N,N-diacetic acid N-(1-carboxyethyl)alanine N-(carboxymethyl)alanine N,N′-methylenebis-glycine N-(aminomethyl)-N-(carboxymethyl)glycine N-(aminomethyl)-N-(carboxymethyl)glycine 2,2′-(methylhydrazono)bis-acetic acid N-(2-carboxyethyl)-N-(4-methylphenyl)-beta-alanine N-(2-carboxyethyl)-N-(3-methylphenyl)-beta-alanine 3-[(carboxymethyl)amino]alanine D-alpha-aspartyl-D-alanine N-(2-carboxyethyl)-N-(1-oxohexadecyl)-beta-alanine N-(2-carboxyethyl)-N-(1-oxodecyl)-beta-alanine N-(2-carboxyethyl)-N-(1-oxotetradecyl)-beta-alanine amino[(carboxymethyl)thio]acetic acid N,N′-1,6-hexanediylbis-beta-alanine N-(carboxymethyl)-N-phenyl-beta-alanine N-(1-carboxyethyl)-L-alanine L-glutamic acid L-aspartic acid.

### Amino Acids

Amino acids are well known. An “amino acid” is any organic compound having both an amino group and a carboxylic acid group. In some cases, the one or more amino acids are N-acyl amino acids. The amino acid can be one of any of the amino acids known in the art. The N-acyl amino acids correspond to the formula:

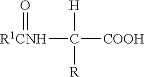

wherein R can be a hydrogen, alkyl (substituted or unsubstituted, branched or straight chain), or a combination of alkyl and aromatic groups. A list of possible side chains of amino acids known in the art are described in Stryer, Biochemistry, 1981, published by W.H. Freeman and Company, which is incorporated herein by reference in its entirety. R¹ can be C1 to C30, saturated or unsaturated, straight or branched, substituted or unsubstituted alkyls; substituted or unsubstituted aromatic groups; or mixtures thereof.

In some cases, the N-acyl amino acid compound is selected from the group consisting of N-acyl Phenylalanine, and N-acyl Tyrosine. The amino acid can be the D or L isomer or a mixture thereof. N-acyl Phenylalanine corresponds to the following formula:

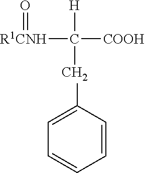

wherein R¹ can be C1 to C30, saturated or unsaturated, straight or branched, substituted or unsubstituted alkyls; substituted or unsubstituted aromatic groups; or mixtures thereof. N-acyl Tyrosine corresponds to the following formula:

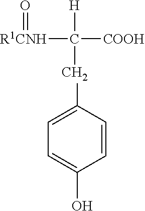

wherein R¹ can be C1 to C30, saturated or unsaturated, straight or branched, substituted or unsubstituted alkyls.

### Cationic Conditioning Compounds (Optional)

The cationic conditioning agent may be used in the compositions of the present invention, and if are present are preferably selected from one or more monoalkyl quaternary amine, such as stearyltrimonium chloride, soyatrimonium chloride or coco-ethyldimonium ethosulfate. Other suitable cationic conditioning agents include, but are not limited to, behentrimonium chloride, dialkyl quaternary amines, such as dicetyldimonium chloride, dicocodimethyl ammonium chloride or distearyldimethyl ammonium chloride; and polyquaternium compounds, such as Polyquaternium-6, Polyquaternium-22 or Polyquaternium-5.

For example, cationic conditioning agents may be chosen from polyquaterium-10 (also called quaternized polyhydroxyethyl cellulose), cetrimonium chloride (also called cetyl trimethyl ammonium chloride, CTAC), behentrimonium chloride (also known as docosyl trimethyl ammonium chloride), behentrimonium methosulfate, steartrimonium chloride, stearalkonium chloride, dicetyldimonium chloride, hydroxypropyltrimonium chloride, cocotrimonium methosulfate, olealkonium chloride, steartrimonium chloride, babassuamidopropalkonium chloride, brassicamidopropyl dimethylamine, Quaternium-91, Salcare/PQ-37, Quaternium-22, Quaternium-87, Polyquaternium-4, Polyquaternium-6, Polyquaternium-11, Polyquaternium-44, Polyquaternium-67, amodimethicone, lauryl betaine, Polyacrylate-1 Crosspolymer, steardimonium hydroxypropyl hydrolyzed wheat protein, behenamidopropyl PG-dimonium chloride, lauryldimonium hydroxypropyl hydrolyzed soy protein, aminopropyl dimethicone, Quaterium-8, and dilinoleamidopropyl dimethylamine dimethicone PEG-7 phosphate.

In some instances, the cationic conditioning agents are cationic conditioning polymers. Examples of cationic conditioning polymers that can be used include, without limitation, cationic cellulose, cationic proteins, and cationic polymers. The cationic polymers can have a vinyl group backbone of amino and/or quaternary ammonium monomers. Cationic amino and quaternary ammonium monomers include, without limitation, dialkylamino alkylmethacrylate, monoalkylaminoalkyl acrylate, monoalkylaminoalkyl methacrylate, trialkyl methacryoloxyalkyl ammonium salt, trialkyl acryloxyalkyl ammonium salts, diallyl quaternary ammonium salts, vinyl compounds substituted with dialkyl aminoalkyl acrylate, and vinyl quaternary ammonium monomers having cyclic cationic nitrogen containing rings such as pyridinium, imidazolium, or quaternized pyrrolidine. Other examples of cationic conditioning polymers that can be used include, without limitation, hydroxypropyltrimonium honey, cocodimonium silk amino acids, cocodimonium hydroxypropyl hydrolyzed wheat or silk protein, polyquaternium-5, polyquaternium-11, polyquaternium-2, polyquaternium-4, polyquaternium-6, polyquaternium-7, polyquaternium-14, polyquaternium-16, polyquaternium-22, polyquaternium-10, and guar hydroxypropyltrimonium chloride.

In some cases, quaternized polymeric cationic conditioning agents are particularly useful. Particularly preferred are quaternary nitrogen polymers prepared by the polymerization of a dialkyldiallylammonium salt or copolymer thereof in which the alkyl group contains 1 to about 18 carbon atoms, and more preferably where the alkyl group is methyl or ethyl. Details concerning the preparation of these polymers can be found in U.S. Pat. Nos. 3,288,770, 3,412,019 and 4,772,462, incorporated herein by reference. For example, cationic homopolymers and copolymers of polydiallyldimethylammonium chloride are available in aqueous compositions sold under the trademark MERQUAT by the Calgon Corporation, subsidiary of Merck & Co., Pittsburgh, Pa. The homopolymer, which is named Polyquaternium-6 in the CTFA Cosmetic Ingredient Dictionary, 3rd Ed., published in 1982 by the Cosmetic Toiletry and Fragrance Association, Inc. (hereafter CTFA Dictionary and CTFA name), is sold under the trademark MERQUAT-100, and is described as having a weight average molecular weight of approximately 100,000. A copolymer reaction product of dimethyldiallylammonium chloride with acrylamide monomers is named Polyquaternium-7 in the CTFA Dictionary, is described as having a weight average molecular weight of approximately 500,000 and is sold under the trademark

MERQUAT-550. Another copolymer reaction product of dimethyldiallylammonium chloride with acrylic acids having a weight average molecular weight from about 50,000 to about 10,000,000 has the CTFA name Polyquaternium-22 and is sold under the trademark MERQUAT-280. Polyquaternium-6 is particularly preferred.

Other polymeric conditioners include cationic copolymers of methylvinylimidazolium chloride and vinyl pyrrolidone, sold commercially by BASF Aktiengesellschaft, West Germany under the trademark LUVIQUAT at three comonomer ratios, namely at ratios of 95/5, 50/50 and 30/70 methylvinylimidazolium chloride to polyvinylpyrrolidone. These copolymers at all three comonomer ratios have the CTFA name Polyquaternium 16. Polymeric conditioners also include cationic cellulosic polymers of hydroxyethyl cellulose reacted with epichlorohydrin and quaternized with trimethylamine, sold under the trademark POLYMER JR in various viscosity grades and molecular sizes by Union Carbide Corporation, Danbury, Conn. These series of polymers are named Polyquaternium 10 in the CTFA Dictionary. Also useful are quaternized copolymers of hydroxyethylcellulose and dimethyldimethylammonium chloride, having the CTFA name Polyquaternium-4, sold in varying molecular weights under the trademark CELQUAT by National Starch and Chemical Corporation, Bridgewater, N.J.

Smaller molecule cationic non-polymeric conditioning agents can also be utilized herein. Exemplary small-molecule conditioning agents can include monofunctional or difunctional quaternary ammonium compounds, such as stearyldimethylbenzylammonium chloride, dimethyldi-(hydrogenated tallow)ammonium chloride, and the like. Non-polymeric conditioning agents can also include the quaternary ammonium salts of gluconamide derivatives, such as gamma-gluconamidopropyldimethyl-2-hydroxyethyl-ammonium chloride and minkamidopropyldimethyl-2-hydroxyethylammonium chloride identified respectively by the CTFA names Quaternium 22 and Quaternium 26. Details for the preparation of these materials are found in U.S. Pat. Nos. 3,766,267 and 4,012,398, respectively, and the materials are sold under the trademark CERAPHYL by Van Dyk & Co., Belleville, N.J. Also useful are bis-quaternary ammonium compounds which are dimers, such as 2-hydroxy propylene-bis-1,3-(dimethylstearyl ammonium chloride, designated the CTFA name, Hydroxypropyl Bisstearyldimonium chloride. The preparation of these and other bis-quat materials is described in U.S. Pat. No. 4,734,277, and such materials are sold under the trademark JORDAQUAT DIMER by Jordan Chemical Company, Folcroft, Pa.

Exemplary unquaternized polymers having tertiary amino nitrogen groups that become quaternized when protonated can include water-soluble proteinaceous quaternary ammonium compounds. Cocodimonium hydrolyzed animal protein, for example, is the CTFA name for a chemically-modified quaternary ammonium derivative of hydrolyzed collagen protein having from about 12 to about 18 carbons in at least one aliphatic alkyl group, a weight average molecular weight from about 2500 to about 12,000, and an isoionic point in a range from about 9.5 to about 11.5. This material and structurally related materials are sold under the trademarks CROQUAT and CROTEIN by Croda, Inc., New York, N.Y.

In some aspects, the subject compositions do not include polymeric conditioners.

### Bleaching Agents

Bleaching agents may be selected from, for example, peroxides, persulfates, perborates percarbonates, alkali metal bromates, ferricyanides, peroxygenated salts, or a mixture thereof. Bleaching agents that may also be used include at least one redox enzyme such as laccases, peroxidases, and 2-electron oxidoreductases, such as uricase, where appropriate in the presence of their respective donor or co-factor. Oxygen in the air may also be employed as an oxidizing agent.

In some cases, the bleaching agent is a persulfate and/or a monopersulfate such as, for example, potassium persulfate, sodium persulfate, ammonium persulfate, as well as mixtures thereof. In one embodiment, the oxidizing agents in the present disclosure are selected from hydrogen peroxide, potassium persulfate, sodium persulfate, and mixtures thereof.

### Developer

The hair lightening compositions of the instant disclosure may be combined with a developer composition. A developer composition is typically an aqueous composition that includes one or more oxidizing agents, such as peroxide. Non-limiting examples of oxidizing include those that are water soluble such as peroxygen oxidizing agents. The oxidizing agent may be selected from water-soluble oxidizing agents which are inorganic peroxygen materials capable of yielding hydrogen peroxide in an aqueous solution.

The oxidizing agents may be provided in aqueous solution or as a powder which is dissolved prior to use. Water-soluble oxidizing agents include hydrogen peroxide, inorganic alkali metal peroxides such as sodium peroxide and sodium peroxide and organic peroxides such as urea peroxide, melamine peroxide, and inorganic perhydrate salt bleaching compounds, such as the alkali metal salts of perborates, percarbonates, perphosphates, persilicates, persulphates and the like. These inorganic perhydrate salts may be incorporated as monohydrates, tetrahydrates etc. Alkyl and aryl peroxides, and or peroxidases may also be used. Mixtures of two or more such oxidizing agents can also be used if desired. In some cases, hydrogen peroxide, percarbonate, persulphates and combinations thereof, are used in the developer compositions.

The oxidative agent may comprise from about 0.1% to about 40% by weight, from about 1% to about 30% by weight, or from about 2% to about 30% by weight of the developer composition. Another potential oxidizing agent for use herein is a source of peroxymonocarbonate ions. Preferably such a source is formed in situ from a source of hydrogen peroxide and a hydrogen carbonate ion source. This system is particularly effective in combination with a source of ammonia or ammonium ions. Accordingly, any source of these peroxymonocarbonate ions may be utilized. Suitable sources for use herein include sodium, potassium, guanidine, arginine, lithium, calcium, magnesium, barium, ammonium salts of carbonate, carbamate and hydrocarbonate ions and mixtures thereof such as sodium carbonate, sodium hydrogen carbonate, potassium carbonate, potassium hydrogen carbonate, guanidine carbonate, guanidine hydrogen carbonate, lithium carbonate, calcium carbonate, magnesium carbonate, barium carbonate, ammonium carbonate, ammonium hydrogen carbonate and mixtures thereof. Percarbonate salts may also be utilized to provide both the source of carbonate ions and as an oxidizing agent. Non-limiting examples of carbonate ions, carbamate and hydrocarbonate ions are sodium hydrogen carbonate, potassium hydrogen carbonate, ammonium carbamate, and mixtures thereof.

### Oils

The one or more oils typically include those generally used in cosmetics and particularly from oils of animal, vegetable or mineral origin, linear or branched hydrocarbons, optionally branched and/or unsaturated fatty acids, optionally branched and/or unsaturated fatty alcohols, mono- and/or polyesters of fatty acids and/or of fatty alcohols, perfluorinated and/or organofluorinated oils, volatile or non-volatile silicone oils, fluorosilicone oils and their mixtures.

Non-limiting examples of oils include oils of animal, vegetable or mineral origin, of lanolin, squalene, fish oil, perhydrosqualene, mink oil, turtle oil, soybean oil, grape seed oil, sesame oil, maize oil, rapeseed oil, sunflower oil, cottonseed oil, avocado oil, olive oil, castor seed oil, jojoba seed oil, peanut oil, sweet almond oil, palm oil, cucumber oil, hazelnut oil, apricot kernel oil, wheat germ oil, calophyllum oil, macadamia oil, coconut oil, cereal germ oil, candlenut oil, thistle oil, candelilla oil, safflower oil, shea butter, and their mixtures.

Mention is made, as an example of linear or branched hydrocarbons, of oils of mineral or synthetic origin, such as liquid paraffins and their derivatives, petrolatum, mineral oils, (mixtures of oil-derived hydrocarbon oils), polybutene, hydrogenated polyisobutene, polyisoprene, polydecenes such as hydrogenated polydecene, or also linear, branched and/or cyclic alkanes which are optionally volatile, such as, for example, isohexadecane, isododecane, isodecane or isohexadecane, and their mixtures.

Mention is made, as examples of optionally branched and/or unsaturated fatty acids, of myristic acid, palmitic acid, stearic acid, behenic acid, oleic acid, linoleic acid, linolenic acid or isostearic acid, and their mixtures.

Mention is made, as example of optionally branched and/or unsaturated fatty alcohols, of cetanol, stearyl alcohol, oleyl alcohol, cetyl alcohol, octyldodecanol, and their mixtures.

Mention is made, as examples of esters, of monoesters or polyesters of fatty acids, the linear or branched fatty chain of which includes from 6 to 30 carbon atoms, and of fatty alcohols, the fatty chain of which includes from 3 to 30 carbon atoms, in particular mono- and polyesters of hydroxy acids and of fatty alcohols, esters of benzoic acid and of fatty alcohols, polyesters of polyols, dipentaerythrityl C₅-C₉ esters, trimethylolpropane polyesters, propylene glycol polyesters, polyesters of hydrogenated castor oil.

Further mention is made of the oils of the group consisting of isononyl isononanoate, stearyl octanoate, isopropyl palmitate, isopropyl myristate, butyl stearate, hexyl laurate, 2-ethylhexyl palmitate, 2-hexyldecyl laurate, 2-octyldecyl palmitate, 2-octyldodecyl myristate, di(2-ethylhexyl) succinate, diisostearyl malate, 2-octyldodecyl lactate, glyceryl triisostearate or diglyceryl triisostearate, octyldodecyl stearoyl stearate (Ceraphyl), cetearyl isononanoate, diisopropyl adipate, caprylic/capric triglyceride, glyceryl tricaprate/caprylate, isocetyl stearoyl stearate, C₁₂-C₁₅ alkyl benzoates, pentaerythrityl tetraisostearate, dipentaerythrityl pentaisononanoate, bis-diglyceryl polyacyladipate-2, trimethylolpropane triethylhexanoate, propylene glycol dibenzoate, propylene glycol dioctanoate, and mixture thereof.

Mention is made, as example of volatile silicone oils, of hexamethyldisiloxane, dimethicones with a viscosity of between 0.65 and 5 mm²/s, octamethylcyclotetrasiloxane, heptamethylhexyltrisiloxane, decamethylcyclopentasiloxane, decamethyltetrasiloxane, octamethyltrisiloxane, dodecamethylcyclohexasiloxane, heptamethyloctyltrisiloxane, and their mixtures.

Mention is made, as example of non-volatile silicone oils, of non-volatile polydialkylsiloxanes; polydimethylsiloxanes comprising pendant alkyl, alkoxy or phenyl groups or alkyl, alkoxy or phenyl groups at the end of the silicone chain, which groups have from 2 to 24 carbon atoms; phenylated silicones, such as those of the phenyl trimethicone type, those of the phenylpropyldimethylsiloxysilicate type or those of the trimethylpentaphenyltrisiloxane type; polysiloxanes modified by fatty acids, in particular C₈-C₂₀ fatty acids, fatty alcohols, in particular C₈-C₂₀ fatty alcohols, or polyoxyalkylenes (in particular polyoxyethylene and/or polyoxypropylene); aminated polysiloxanes; polysiloxanes comprising a hydroxyl group; and their mixtures.

Mention is made, as fluorosilicone oils, of fluorinated polysiloxanes comprising a pendant fluorinated group or a fluorinated group at the end of the silicone chain having from 1 to 12 carbon atoms, all or a portion of the hydrogens of which are replaced by fluorine atoms, such as perfluorononyl dimethicone, and their mixtures.

### Thickening Agents

The compositions may contain one or more thickeners or viscosity modifying agents. Classes of such agents include, but are not limited to, viscous liquids, such as polyethylene glycol, semisynthetic polymers, such as semisynthetic cellulose derivatives, synthetic polymers, such as carbomers, poloxamers, and polyethyleneimines (e.g., PEI-10), naturally occurring polymers, such as acacia, tragacanth, alginates (e.g., sodium alginate), carrageenan, vegetable gums, such as xanthan gum, petroleum jelly, waxes, particulate associate colloids, such as bentonite, colloidal silicon dioxide, and microcrystalline cellulose, surfactants, such as PPG-2 hydroxyethyl coco/isostearamide, emulsifiers, such as disteareth-75 IPDI, and salts, such as sodium chloride, and combinations thereof.

### Colorants

Before, after, or simultaneously with the hair lightening composition, a color-altering composition may be used. For example, the color-altering composition may be formed by combining a hair lightening composition according to the instant disclosure, a developer composition (typically comprising hydrogen peroxide) and a colorant. Typically, the coloring compositions of the present disclosure include at least one colorant compound chosen from oxidative dye precursors, direct dyes, pigments, and mixtures thereof. The oxidation dyes are generally chosen from one or more oxidation bases optionally combined with one or more couplers. By way of example, the oxidation bases are chosen from para-phenylenediamines, bis(phenyl)alkylenediamines, para-aminophenols, ortho-aminophenols and heterocyclic bases, and the addition salts thereof.

Among the para-phenylenediamines that may be mentioned, for example, are para-phenylenediamine, para-toluenediamine, 2-chloro-para-phenylenediamine, 2,3-dimethyl-para-phenylenediamine, 2,6-dimethyl-para-phenylenediamine, 2,6-diethyl-para-phenylenediamine, 2,5-dimethyl-para-phenylenediamine, N,N-dimethyl-para-phenylenediamine, N,N-diethyl-para-phenylenediamine, N,N-dipropyl-para-phenylenediamine, 4-amino-N,N-diethyl-3-methylaniline, N,N-bis(.beta.-hydroxyethyl)-para-phenylenediamine, 4-N, N-bis(.beta.-hydroxyethyl)amino-2-methylaniline, 4-N,N-bis(.beta.-hydroxyethyl)amino-2-chloroaniline, 2-.beta.-hydroxyethyl-para-phenylenediamine, 2-methoxymethyl-para-phenylenediamine, 2-fluoro-para-phenylenediamine, 2-isopropyl-para-phenylenediamine, N-(.beta.-hydroxypropyl)-para-phenylenediamine, 2-hydroxymethyl-para-phenylenediamine,

N,N-dimethyl-3-methyl-para-phenylenediamine, N-ethyl-N-(.beta.-hydroxyethyl)-para-phenylenediamine, N-(.beta.,.gamma.-dihydroxypropyl-para-phenylenediamine, N-(4′-aminophenyl)-para-phenylenediamine, N-phenyl-para-phenylenediamine, 2-.beta.-hydroxyethyloxy-para-phenylenediamine, 2-.beta.-acetylaminoethyloxy-para-phenylenediamine, N-(.beta.-methoxyethyl)-para-phenylenediamine, 4-aminophenylpyrrolidine, 2-thienyl-para-phenylenediamine, 2-.beta.-hydroxyethylamino-5-aminotoluene and 3-hydroxy-1-(4′-aminophenyl)pyrrolidine, and the addition salts thereof with an acid.

Among the para-phenylenediamines mentioned above, para-phenylenediamine, para-toluenediamine, 2-isopropyl-para-phenylenediamine, 2-.beta.-hydroxyethyl-para-phenylenediamine, 2-.beta.-hydroxyethyloxy-para-phenylenediamine, 2,6-dimethyl-para-phenylenediamine, 2,6-diethyl-para-phenylenediamine, 2,3-dimethyl-para-phenylenediamine, N,N-bis(.beta.-hydroxyethyl)-para-phenylenediamine, 2-chloro-para-phenylenediamine and 2-.beta.-acetylaminoethyloxy-para-phenylenediamine, and the addition salts thereof with an acid, are particularly preferred.

Among the bis(phenyl)alkylenediamines that may be mentioned, for example, are N,N′-bis(.beta.-hydroxyethyl)-N,N′-bis(4′-aminophenyl)-1,3-diaminopropano-I, N,N′-bis(.beta.-hydroxyethyl)-N,N′-bis(4′-aminophenyl)ethylenediamine, N,N′-bis(4-aminophenyl)tetramethylenediamine, N,N′-bis(.beta.-hydroxyethyl)-N,N′-bis(4-aminophenyl)tetramethylenediamin-e, N,N′-bis(4-methylaminophenyl)tetramethylenediamine, N,N′-bis(ethyl)-N,N′-bis(4′-amino-3′-methylphenyl)ethylenediamine and 1,8-bis(2,5-diaminophenoxy)-3,6-dioxaoctane, and the addition salts thereof.

Among the para-aminophenols that may be mentioned, for example, are para-aminophenol, 4-amino-3-methylphenol, 4-amino-3-fluorophenol, 4-amino-3-chlorophenol, 4-amino-3-hydroxymethylphenol, 4-amino-2-methylphenol, 4-amino-2-hydroxymethylphenol, 4-amino-2-methoxymethylphenol, 4-amino-2-aminomethyl phenol, 4-amino-2-(.beta.-hydroxyethylaminomethyl)phenol and 4-amino-2-fluorophenol, and the addition salts thereof with an acid.

Among the ortho-aminophenols that may be mentioned, for example, are 2-aminophenol, 2-amino-5-methylphenol, 2-amino-6-methylphenol and 5-acetamido-2-aminophenol, and the addition salts thereof.

Among the heterocyclic bases that may be mentioned, for example, are pyridine derivatives, pyrimidine derivatives and pyrazole derivatives.

Among the pyridine derivatives that may be mentioned are the compounds such as 2,5-diaminopyridine, 2-(4-methoxyphenyl)amino-3-aminopyridine and 3,4-diaminopyridine, and the addition salts thereof. Other pyridine oxidation bases that are useful in the present disclosure are the 3-aminopyrazolo[1,5-a]pyridine oxidation bases or the addition salts thereof described, for example, in patent application FR 2 801 308. Examples that may be mentioned include pyrazolo[1,5-a]pyrid-3-ylamine, 2-acetylaminopyrazolo[1,5-a]pyrid-3-ylamine, 2-morpholin-4-ylpyrazolo[1,5-a]pyrid-3-ylamine, 3-aminopyrazolo[1,5-a]pyridine-2-carboxylic acid, 2-methoxypyrazolo[1,5-a]pyrid-3-ylamine, (3-aminopyrazolo[1,5-a]pyrid-7-yl)methanol, 2-(3-aminopyrazolo[1,5-a]pyrid-5-yl)ethanol, 2-(3-aminopyrazolo[1,5-a]pyrid-7-yl)ethanol, (3-aminopyrazolo[1,5-a]pyrid-2-yl)methanol, 3,6-diaminopyrazolo[1,5-a]pyridine, 3,4-diaminopyrazolo[1,5-a]pyridine, pyrazolo[1,5-a]pyridine-3,7-diamine, 7-morpholin-4-ylpyrazolo[1,5-a]pyrid-3-ylamine, pyrazolo[1,5-a]pyridine-3,5-diamine, 5-morpholin-4-ylpyrazolo[1,5-a]pyrid-3-ylamine, 2-[(3-aminopyrazolo[1,5-a]pyrid-5-yl)(2-hydroxyethyl)amino]ethanol, 2-[(3-aminopyrazolo[1,5-a]pyrid-7-yl)(2-hydroxyethyl)amino]ethanol, 3-aminopyrazolo[1,5-a]pyridin-5-ol, 3-aminopyrazolo[1,5-a]pyridin-4-ol, 3-aminopyrazolo[1,5-a]pyridin-6-ol, 3-aminopyrazolo[1,5-a]pyridin-7-ol, 2-.quadrature.-hydroxyethoxy-3-amino-pyrazolo[1,5-a]pyridine; 2-(4-dimethylpyperazinium-1-yl)-3-amino-pyrazolo[1,5-a]pyridine; and the addition salts thereof.

More particularly oxidation bases that are useful in the present disclosure are selected from 3-aminopyrazolo-[1,5-a]-pyridines and preferably substituted on carbon atom 2 by:

(a) one (di)(C₁-C₆)(alkyl)amino group wherein said alkyl group can be substituted by at least one hydroxy, amino, imidazolium group;

(b) one heterocycloalkyl group containing from 5 to 7 members chain, and from 1 to 3 heteroatoms, potentially cationic, potentially substituted by one or more (C₁-C₆-alkyl, such as di(C1-C4)alkylpiperazinium; or

(c) one (C₁-C₆)alkoxy potentially substituted by one or more hydroxy groups such as .quadrature.-hydroxyalkoxy, and the addition salts thereof.

Among the pyrimidine derivatives that may be mentioned are compounds such as 2,4,5,6-tetraaminopyrimidine, 4-hydroxy-2,5,6-triaminopyrimidine, 2-hydroxy-4,5,6-triaminopyrimidine, 2,4-dihydroxy-5,6-diaminopyrimidine, 2,5,6-triaminopyrimidine and their addition salts and their tautomeric forms, when a tautomeric equilibrium exists.

Among the pyrazole derivatives that may be mentioned are compounds such as 4,5-diamino-1-methyl-pyrazole, 4,5-diamino-1-(.beta.-hydroxyethyl)pyrazole, 3,4-diamino-pyrazole, 4,5-diamino-1-(4′-chlorobenzyl)pyrazole, 4,5-diamino-1,3-dimethylpyrazole, 4,5-diamino-3-methyl-1-phenyl-pyrazole, 4,5-diamino-1-methyl-3-phenylpyrazole, 4-amino-1,3-dimethyl-5-hydrazinopyrazole, 1-benzyl-4,5-diamino-3-methyl-pyrazole, 4,5-diamino-3-tert-butyl-1-methylpyrazole, 4,5-diamino-1-tert-butyl-3-methylpyrazole, 4,5-diamino-1-(.beta.-hydroxyethyl)-3-methylpyrazole, 4,5-diamino-1-ethyl-3-methyl-pyrazole, 4,5-diamino-1-ethyl-3-(4′-methoxyphenyl)pyrazole, 4,5-diamino-1-ethyl-3-hydroxymethylpyrazole, 4,5-diamino-3-hydroxymethyl-1-methylpyrazole, 4,5-diamino-3-hydroxymethyl-1-isopropylpyrazole, 4,5-diamino-3-methyl-1-isopropylpyrazole, 4-amino-5-(2′-aminoethyl)amino-1,3-dimethylpyrazole, 3,4,5-triaminopyrazole, 1-methyl-3,4,5-triaminopyrazole, 3,5-diamino-1-methyl-4-methylaminopyrazole, 3,5-diamino-4-(.beta.-hydroxyethyl)amino-1-methylpyrazole, and the addition salts thereof. 4,5-Diamino-1-(.beta.-methoxyethyl)pyrazole may also be used.

Pyrazole derivatives that may also be mentioned include diamino-N,N-dihydropyrazolopyrazolones such as the following compounds and the addition salts thereof: 2,3-diamino-6,7-dihydro-1H,5H-pyrazolo[1,2-a]pyrazol-1-one, 2-amino-3-ethylamino-6,7-di hydro-1H,5H-pyrazolo[1,2-a]pyrazol-1-one, 2-amino-3-isopropylamino-6,7-dihydro-1H,5H-pyrazolo[1,2-a]pyrazol-1-one, 2-amino-3-(pyrrolidin-1-yl)-6,7-dihydro-1H,5H-pyrazolo[1,2-a]pyrazol-1-on- e, 4,5-diamino-1,2-dimethyl-1,2-dihydropyrazol-3-one, 4,5-diamino-1,2-diethyl-1,2-dihydropyrazol-3-one, 4,5-diamino-1,2-di-(2-hydroxyethyl)-1,2-dihydropyrazol-3-one, 2-amino-3-(2-hydroxyethyl)amino-6,7-dihydro-1H,5H-pyrazolo[1,2-a]pyrazol-- 1-one, 2-amino-3-dimethylamino-6,7-dihydro-1H,5H-pyrazolo[1,2-a]pyrazol-1-one, 2,3-diamino-5,6,7,8-tetrahydro-1H,6H-pyridazino[1,2-a]pyrazol-1-one, 4-amino-1,2-diethyl-5-(pyrrolidin-1-yl)-1,2-dihydropyrazol-3-one, 4-amino-5-(3-dimethylaminopyrrolidin-1-yl)-1,2-diethyl-1,2-dihydropyrazol-3-one, 2,3-diamino-6-hydroxy-6,7-dihydro-1H,5H-pyrazolo[1,2-a]pyrazol-1-o-ne. 2,3-Diamino-6,7-dihydro-1H,5H-pyrazolo[1,2-a]pyrazol-1-one and/or a salt thereof will preferably be used. 4,5-Diamino-1-(.beta.-hydroxyethyl)pyrazole and/or 2,3-diamino-6,7-dihydro-1H,5H-pyrazolo[1,2-a]pyrazol-1-one and/or a salt thereof will preferentially be used as heterocyclic bases.

Composition according to the present disclosure may optionally comprise one or more couplers advantageously chosen from those conventionally used in the dyeing or coloring of keratin fibers.

Among these couplers, mention may be made especially of meta-phenylenediamines, meta-aminophenols, meta-diphenols, naphthalene-based couplers and heterocyclic couplers, and also the addition salts thereof.

Mention may be made, for example, of 2-methyl-5-aminophenol, 5-N-(.beta.-hydroxyethyl)amino-2-methylphenol, 3-aminophenol, 5-amino-6-chloro-o-cresol (3-amino-2-chloro-6-methylphenol), 1,3-dihydroxybenzene, 1,3-dihydroxy-2-methyl-benzene, 4-chloro-1,3-dihydroxybenzene, 2,4-diamino-1-(.beta.-hydroxyethyloxy)benzene, 2-amino-4-(.beta.-hydroxyethylamino)-1-methoxybenzene, 1,3-diaminobenzene, 1,3-bis(2,4-diamino-phenoxy)propane, 3-ureidoaniline, 3-ureido-1-dimethylamino-benzene, sesamol, 1-.beta.-hydroxyethylamino-3,4-methylene-dioxybenzene, .alpha.-naphthol, 2-methyl-1-naphthol, 6-hydroxyindole, 4-hydroxyindole, 4-hydroxy-N-methylindole, 2-amino-3-hydroxypyridine, 6-hydroxybenzomorpholine, 3,5-diamino-2,6-dimethoxypyridine, 1-N-(.beta.-hydroxyethyl)amino-3,4-methylene-dioxybenzene, 2,6-bis(J-hydroxyethylamino)toluene, 6-hydroxy-indoline, 2,6-dihydroxy-4-methylpyridine, 1-H-3-methylpyrazol-5-one, 1-phenyl-3-methylpyrazol-5-one, 2,6-dimethyl-pyrazolo[1,5-b]-1,2,4-triazole, 2,6-dimethyl[3,2-c]-1,2,4-triazole and 6-methylpyrazolo[1,5-a]benzimidazole, the addition salts thereof with an acid, and mixtures thereof.

In general, the addition salts of the oxidation bases and couplers that may be used in the context of the disclosure are especially selected from the addition salts with an acid such as the hydrochlorides, hydrobromides, sulfates, citrates, succinates, tartrates, lactates, tosylates, benzenesulfonates, phosphates and acetates.

The oxidation base(s) each advantageously represent from 0.001% to 10% by weight relative to the total weight of the composition, and preferably from 0.005% to 5% by weight relative to the total weight of the compositions of the present disclosure.

The coupler(s), if they are present, each advantageously represent from 0.001% to 10% by weight relative to the total weight of the composition, and preferably from 0.005% to 5% by weight relative to the total weight of the compositions of the present disclosure.

Compositions according to the disclosure may optionally comprise b) one or more synthetic or natural direct dyes, chosen from anionic and nonionic species, preferably cationic or nonionic species, either as sole dyes or in addition to the oxidation dye(s).

Examples of suitable direct dyes that may be mentioned include azo direct dyes; (poly)methine dyes such as cyanins, hemicyanins and styryls; carbonyl dyes; azine dyes; nitro(hetero)aryl dyes; tri(hetero)arylmethane dyes; porphyrin dyes; phthalocyanin dyes, and natural direct dyes, alone or as mixtures.

Many direct dyes are cationic direct dyes. Mention may be made of the hydrazono cationic dyes of formulas (Va) and (V′a), the azo cationic dyes (VIa) and (VI′a) and the diazo cationic dyes (VIIa) below:

| | |
|---|---|
| Het⁺—C(R^{a})═N—N(R^{b})—Ar, An⁻ | (Va) |
| Het⁺—N(R^{a})—N═C(R^{b})—Ar, An⁻ | (V′a) |
| Het⁺—N═N—Ar, An⁻ | (VIa) |
| Ar⁺—N═N—Ar″, An⁻ | (VI′a) and |
| Het⁺—N═N—Ar′—N═N—Ar, An⁻ | (VIIa) |

in which formulas (Va), (V′a), (VIa), (VI′a) and (VIIa):
Het⁺ represents a cationic heteroaryl radical, preferably bearing an endocyclic cationic charge, such as imidazolium, indolium or pyridinium, optionally substituted preferentially with one or more (C₁-C₈) alkyl groups such as methyl;
Ar⁺ representing an aryl radical, such as phenyl or naphthyl, bearing an exocyclic cationic charge, preferentially ammonium, particularly tri(C₁-C₈)alkylammonium such as trimethylammonium;
Ar represents an aryl group, especially phenyl, which is optionally substituted, preferentially with one or more electron-donating groups such as i) optionally substituted (C₁-C₈)alkyl, ii) optionally substituted (C₁-C₈)alkoxy, iii) (di)(C₁-C₈)(alkyl)amino optionally substituted on the alkyl group(s) with a hydroxyl group, iv) aryl(C₁-C₈)alkylamino, v) optionally substituted N—(C₁-C₈)alkyl-N-aryl(C₁-C₈)alkylamino or alternatively Ar represents a julolidine group;
Ar′ is an optionally substituted divalent (hetero)arylene group such as phenylene, particularly para-phenylene, or naphthalene, which are optionally substituted, preferentially with one or more groups (C₁-C₈)alkyl, hydroxyl or (C₁-C₈)alkoxy
Ar″ is an optionally substituted (hetero)aryl group such as phenyl or pyrazolyl, which are optionally substituted, preferentially with one or more groups (C₁-C₈)alkyl, hydroxyl, (di)(C₁-C₈)(alkyl)amino, (C₁-C₈)alkoxy or phenyl;
R^{a} and R^{b}, which may be identical or different, represent a hydrogen atom or a group (C₁-C₈)alkyl, which is optionally substituted, preferentially with a hydroxyl group;
or alternatively the substituent R^{a} with a substituent of Het⁺ and/or R_{b} with a substituent of Ar and/or R^{a} with R_{b} form, together with the atoms that bear them, a (hetero)cycloalkyl;
particularly, R^{a} and R_{b} represent a hydrogen atom or a group (C₁-C₄)alkyl, which is optionally substituted with a hydroxyl group;
An⁻ represents an anionic counter-ion such as mesylate or halide. In particular, mention may be made of the azo and hydrazono cationic dyes bearing an endocyclic cationic charge of formulae (Va), (V′a) and (VIa) as defined previously. More particularly those of formulae (Va), (V′a) and (VIa) derived from the dyes described in patent applications WO 95/15144, WO 95/01772 and EP-714954, which are incorporated herein by reference in their entirety.

In some cases, the cationic part is derived from the following derivatives:

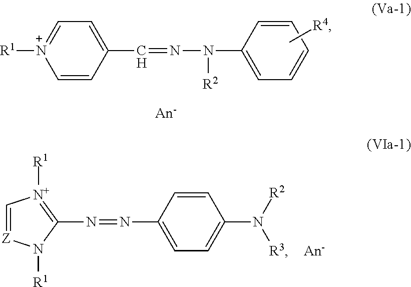

wherein in formulae (Va-1) and (VIa-1):
R¹ representing a (C₁-C₄) alkyl group such as methyl;
R² and R³, which are identical or different, represent a hydrogen atom or a (C₁-C₄)alkyl group, such as methyl; and
R⁴ represents a hydrogen atom or an electron-donating group such as optionally substituted (C₁-C₈)alkyl, optionally substituted (C₁-C₈)alkoxy, or (di)(C₁-C₈)(alkyl)amino optionally substituted on the alkyl group(s) with a hydroxyl group; particularly, R⁴ is a hydrogen atom,
Z represents a CH group or a nitrogen atom, preferentially CH;
An⁻ represents an anionic counter-ion such as mesylate or halide.

Particularly, the dye of formulae (Va-1) and (VIa-1) is chosen from Basic Red 51, Basic Yellow 87 and Basic Orange 31 or derivatives thereof:

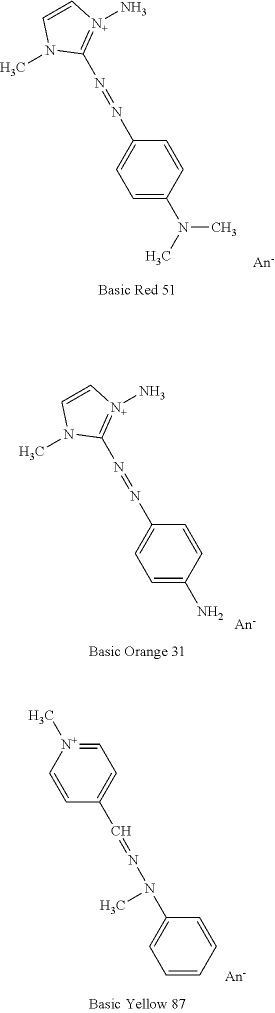

Among the natural direct dyes that may be used according to the disclosure, mention may be made of lawsone, juglone, alizarin, purpurin, carminic acid, kermesic acid, purpurogallin, protocatechaldehyde, indigo, isatin, curcumin, spinulosin, apigenidin and orceins. Extracts or decoctions containing these natural dyes and in particular henna-based poultices or extracts may also be used.

When they are present, the direct dye(s) more particularly represent from 0.001% to 10% by weight and preferably from 0.005% to 5% by weight of the total weight of the compositions of the present disclosure.

### Preservatives

One or more preservatives may be included in the compositions described herein for treating hair. Suitable preservatives include, but are not limited to, glycerin containing compounds (e.g., glycerin or ethylhexylglycerin or phenoxyethanol), benzyl alcohol, parabens (methylparaben, ethylparaben, propylparaben, butylparaben, isobutylparaben, etc.), sodium benzoate, ethylenediamine-tetraacetic acid (EDTA), potassium sorbate, and/or grapefruit seed extract, or combinations thereof. More than one preservative may be included in the composition. Other preservatives are known in the cosmetics industries and include salicylic acid, DMDM Hydantoin, Formaldahyde, Chlorphenism, Triclosan, Imidazolidinyl Urea, Diazolidinyl Urea, Sorbic Acid, Methylisothiazolinone, Sodium Dehydroacetate, Dehydroacetic Acid, Quaternium-15, Stearalkonium Chloride, Zinc Pyrithione, Sodium Metabisulfite, 2-Bromo-2-Nitropropane, Chlorhexidine Digluconate, Polyaminopropyl biguanide, Benzalkonium Chloride, Sodium Sulfite, Sodium Salicylate, Citric Acid, Neem Oil, Essential Oils (various), Lactic Acid, and Vitamin E (tocopherol).

The preservative is optionally included in an amount ranging from about 0.01 wt. % to about 5 wt. %, about 0.15% to about 1 wt. %, or about 1 wt. % to about 3 wt. %, based on the total weight of the composition.

Suitable components, such as those listed above, are included or excluded from the hair care formulations depending on the specific combination of other components, the form of the compositions, and/or the use of the formulation (e.g., hair spray, cream, conditioner, etc.).

### Forms

The compositions described herein may be in any suitable physical form. Suitable forms include, but are not limited to low to moderate viscosity liquids, lotions, milks, mousses, sprays, gels, creams, conditioners, and the like, as well as powders.

i. Spray

The compositions described herein for treating hair may be in the form of a spray. The spray typically includes the composition comprising the one or more lactones and a cosmetically acceptable carrier. In some embodiments, the carrier is water or a water and alcohol mixture. The spray formulation optionally includes an antioxidant, sunscreen agent, vitamin, protein, peptide, plant extract, humectant, oil, emollient, lubricant, thickener, hair conditioning agent, polymer, and/or surfactant. Preferably, the spray formulation includes a preservative. In some embodiments, the formulation includes a fragrance. In some cases, the compositions described herein include a surfactant. In some cases, the compositions described herein contain water, fragrance, a preservative, and one or more lactones. In some cases, the compositions described herein contain water, fragrance, a preservative, and one or more lactones.

In some cases, the composition comprising one or more lactones contains water, a preservative, fragrance, the one or more lactones, and an anti-static agent. In some cases, the compositions described herein contain water, a preservative, fragrance, the one or more lactones, and a hair conditioning agent. In some cases, the compositions described herein contain water, a preservative, fragrance, the one or more lactones, and a surfactant.

The hair spray formulations may be dispensed from containers that include aerosol dispensers or pump spray dispensers. Such dispensers are known in the art and are commercially available from a variety of manufacturers.

When the hair spray formulation is dispensed from a pressurized aerosol container, a propellant may be used to force the composition out of the container. Suitable propellants include, but are not limited to, a liquefiable gas or a halogenated propellant. Examples of suitable propellants include dimethyl ether and hydrocarbon propellants such as propane, n-butane, iso-butane, CFCs, and CFC-replacement propellants. The propellants may be used singly or admixed.

The amount of propellant may range from about 10% to about 60% by weight of the formulation. The propellant may be separated from the hair repair formulation as in a two compartment container. Other suitable aerosol dispensers are those characterized by the propellant being compressed air, which can be filled into the dispenser using a pump or equivalent device prior to use. Conventional non-aerosol pump spray dispensers, i.e., atomizers, may also be used to apply the hair strengthening formulation to the hair.

ii. Conditioners

The compositions disclosed herein for treatment of hair may be in the form of a conditioner. The conditioner typically includes the composition comprising the one or more lactones in a suitable carrier. Additionally, the conditioner may include cationic polymers derived from polysaccharides, for example cationic cellulose derivatives, cationic starch derivatives, cationic guar derivatives and cationic locust bean gum derivatives, synthetic cationic polymers, mixtures or combinations of these agents. The formulation may comprise other synthetic or natural polymers or polymers derived from biological preparation processes, which are functionalized, where appropriate, for example with cationic or neutral groups. These polymers may have a stabilizing or strengthening action on the compositions, and/or a conditioning action (deposition on the surface of the skin or the hair).

iii. Creams

The compositions disclosed herein for treatment of hair may be in the form of a cream. The cream typically includes one or more lactones in a suitable carrier. The one or more lacatones may be included in any suitable concentration. Typical concentrations of the one or more lactones in the cream range from small amounts such as approximately about 0.01% (wt), at least 0.1% (wt), to large amounts, such as up to about 50% (wt).

Implementation of the present disclosure is provided by way of the following examples. The examples serve to illustrate the technology without being limiting in nature. The following Examples are intended to be non-restrictive and explanatory only, with the scope of the invention being defined by the claims.

The ingredient amounts/concentrations in the treatment compositions/formulas described below, unless otherwise indicated, are expressed in % by weight, based on the total weight of the composition/formula comprising the antioxidant composition and the bleach component of the color altering system.

### EXAMPLES

The composition herein may be in the form of an additive adapted to be added to a bleach composition (Ex. A, below). Alternatively, the composition may be a built-in composition, wherein the subject composition is premixed with a bleach composition (Ex. B-Ex. C, below).

TABLE 1
Example: Ex. A-Ex.C: Subject Inventive Formulations (built-in)
with Bleach Composition

| INCI US | Ex. A | Ex. B | Ex. C |
|---|---|---|---|
| Citric Acid | 3.6 | 3.6 | 3.6 |
| Glycine | 0.6 | 0.6 | 0.6 |
| Scutellaria Baicalensis | 0.7 | 0.3 | 0.1 |
| Root Extract | | | |
| Filler | — | — | — |
| Magnesium Carbonate | 9.0 | 9.0 | 9.0 |
| Hydroxide | | | |
| Sodium Silicate | 17 | 17 | 17 |
| Disodium EDTA | ~1 | ~1 | ~1 |
| Sodium Metasilicate | 2.4 | 2.4 | 2.4 |
| Surfactants | ≥10 | ≥10 | ≥10 |
| Potassium Persulfate | 50 | 50 | 50 |
| Ammonium Persulfate | 5.0 | 5.0 | 5.0 |
| Additives/Optional Ingredients* | ≥4.0 | ≥4.0 | ≥4.0 |

*i.e. one or more of vitamins, protein or hydrolyzed protein, preservatives, salts, oils, thickeners, conditioning agents, plant extracts, plant extract derivatives, fragrances, anti-static agents, etc.

TABLE 2
Example: Ex. D: Comparative Formulation
with Bleach Composition

| INCI US | Ex. D |
|---|---|
| Citric Acid | 3.6 |
| Glycine | 0.6 |
| Scutellaria Baicalensis Root Extract | — |
| Filler | — |
| Magnesium Carbonate Hydroxide | 9.0 |
| Sodium Silicate | 17 |
| Disodium EDTA | ~1 |
| Sodium Metasilicate | 2.4 |
| Surfactants | ≥0.8 |
| Potassium Persulfate | 50 |
| Ammonium Persulfate | 5.0 |
| Additives/Optional Ingredients* | ≥4.0 |

*i.e. one or more of vitamins, protein or hydrolyzed protein, preservatives, salts, oils, thickeners, conditioning agents, plant extracts, plant extract derivatives, fragrances, anti-static agents, etc.

TABLE 3
Developer Formulations

| INCI US | 30V Developer | 40V Developer |
|---|---|---|
| Pentasodium Pentetate | 0.15 | 0.15 |
| Hydrogen Peroxide | 18 | 24 |
| Sodium Stannate | 0.04 | 0.04 |
| Phosphoric Acid | — | — |
| Tetrasodium Pyrophosphate | 0.02 | 0.02 |
| Water | 77.59 | 71.59 |
| Glycerine | 0.5 | 0.5 |
| Trideceth-2-Carboxamide Mea | 0.85 | 0.85 |
| Cetearyl Alcohol (and) Ceteareth | 2.85 | 2.85 |

The inventive and comparative formulas (per Tables 1 and 2) were mixed with the bleach composition (bleach powder) and bleach developer (either 30 or 40V) according to the weight amounts provided in Table 3.

The hair lightening compositions of Ex. A-Ex. D were mixed with a 40V developer compositions (comprising hydrogen peroxide) in a mix ratio of 1:2 (hair lightening composition: developer composition) and used to treat hair. The hair was subsequently subjected to cysteic acid analysis, which is a measurement showing the degree of damage to the hair.

The bleach mixtures of Ex. A-Ex. D were applied to Regular Bleach Swatches, obtained from International Hair Import Products, for 50 plus minutes based on reaching equivalent lightening compared to the control. The swatches are rinsed and shampooed once before being blown dry.

Cysteic acid analysis was measure, showing the degree of damage to the hair. Extensive studies of the influence of the cysteine content on the denaturing of the α-helices in keratins have shown, for example, that the melting temperature (transition temperature) of the keratin increases linearly with the cysteine content. The increased stability of the matrix region attributable to the relatively high degree of crosslinking of the high proportion of disulfide bridges in the matrix means that transition of the helices embedded in this matrix is made difficult and, accordingly, results in an increase in the melting temperature. Conversely, a reduction in the melting point and, above all, in the enthalpy of fusion can generally be observed in chemically treated hair.

Elastic modulus (Young's modulus) and break stress were also measured to characterize hair and the degree damage to hair. The elastic modulus represents a measure of the hair's spring-like structure (elasticity). Higher elastic modulus shows that the hair is more elastic (less brittle). Break stress represents the force/area needed to break the hair fiber. A higher break stress represents a stronger and stiffer hair fiber.

Differential Scanning calorimetry (DCS) is a tool that can also be useful for investigating the structural characteristics of hair fibers. Keratin undergoes detectable transformations at various temperatures. Changes in these transformation temperatures can be used to estimate how a particular hair-treatment may impact hair fibers. In the instant case, DSC was used to measure denaturation temperature (T_{d}). Denaturation temperature (T_{d}) has been used as a representation of the thermal stability of hair fibers, which is influenced, at least in part, by the cross-link density of the matrix (intermediate filament associated proteins, IFAP). Thermal stability (T_{d}) and its relationship in determining the thermal stability of hair fibers is established in the literature.

Results: The system was evaluated using miniature tensile testing (MTT) to measure the impact of the bleaching on tensile strength and elastic modulus. Amino acid analysis (AAA) was performed on bleached hair fibers to measure the formation of citric acid, which indicates the amount of cysteine bonds broken. Cosmetic hair attributes were evaluated by professional stylists in comparative on-head testing. Stability was evaluated based on standard procedure. The results for the cysteic acid testing, elastic modulus, break stress, and DSC for hair treated with the hair lightening compositions for Ex. A-Ex. C demonstrated significant improvement in cysteic acid testing, elastic modulus, break stress, and DSC for hair treated with the hair lightening compositions in comparison to that treated with Ex. D. The baicalin/citric acid/glycine combination provides an instrumental benefit to fiber integrity during hair bleaching, resulting in improved tensile strength and elastic modulus, and reduced formation of citric acid, versus the same bleach without the actives. Additionally, the samples with Ex. A-Ex. C were found to provide a lasting smoothness and manageability to the hair after bleaching. The actives of Ex. A-Ex. C found stable in 45° C. chamber, finding no clumping occurred when mixed or built-in to the bleach. Ex. A-Ex. C were evaluated through MTT, AAA, and in salon testing. Compared to Ex. D, Ex. A-Ex. C were found to have a higher tensile strength, higher elastic modulus, and lower presence of cysteic acid. In salon testing, the formula Ex. A-Ex. C was found to provide smoothing and manageability attributes to the volunteers evaluated.

The foregoing description illustrates and describes the disclosure. Additionally, the disclosure shows and describes only the preferred embodiments but, as mentioned above, it is to be understood that it is capable to use in various other combinations, modifications, and environments and is capable of changes or modifications within the scope of the invention concepts as expressed herein, commensurate with the above teachings and/or the skill or knowledge of the relevant art. The embodiments described herein above are further intended to explain best modes known by applicant and to enable others skilled in the art to utilize the disclosure in such, or other, embodiments and with the various modifications required by the particular applications or uses thereof. Accordingly, the description is not intended to limit the invention to the form disclosed herein. Also, it is intended to the appended claims be construed to include alternative embodiments.

As used herein, the terms “comprising,” “having,” and “including” are used in their open, non-limiting sense.

The terms “a,” “an,” and “the” are understood to encompass the plural as well as the singular.

The expression “one or more” means “at least one” and thus includes individual components as well as mixtures/combinations.

“Antioxidant” refers to a chemical compound, an enzyme or other organic molecule which prevents free radicals from causing oxidation of molecules such as are found in keratinous materials. The antioxidant, by reacting with the oxidant, protects such molecules from being damaged. Examples of antioxidants include without limitation, polyphenols, vitamins A, C, E, carotenoids, and certain minerals. In some specific examples, antioxidants include ascorbic acid, dihydrochalone, zinc PCA, baicalin, ferulic acid, pine bark extract, polydatin and ellagic acid.

Other than in the operating examples, or where otherwise indicated, all numbers expressing quantities of ingredients and/or reaction conditions are to be understood as being modified in all instances by the term “about,” meaning within +/−5% of the indicated number.

All percentages, parts and ratios herein are based upon the total weight of the compositions of the present invention, unless otherwise indicated.

“Keratinous substrates” as used herein, includes, but is not limited to keratin fibers such as hair and/or scalp on the human head.

“Conditioning” as used herein means imparting to one or more hair fibers at least one property chosen from combability, moisture-retentivity, luster, shine, and softness. The state of conditioning can be evaluated by any means known in the art, such as, for example, measuring, and comparing, the ease of combability of the treated hair and of the untreated hair in terms of combing work (gm-in), and consumer perception.

The term “treat” (and its grammatical variations) as used herein refers to the application of the compositions of the present disclosure onto the surface of keratinous substrates such as hair. The term “treat” (and its grammatical variations) as used herein also refers to contacting keratinous substrates such as hair with the compositions of the present disclosure.

A “rinse-off” product refers to a composition such as a hair care composition that is rinsed and/or washed with water either after or during the application of the composition onto the keratinous substrate, and before drying and/or styling said keratinous substrate. At least a portion of the composition is removed from the keratinous substrate during the rinsing and/or washing.

The term “stable” as used herein means that the composition does not exhibit phase separation and/or crystallization.

“Volatile”, as used herein, means having a flash point of less than about 100° C.

“Non-volatile”, as used herein, means having a flash point of greater than about 100° C.

As used herein, all ranges provided are meant to include every specific range within, and combination of sub ranges between, the given ranges. Thus, a range from 1-5, includes specifically 1, 2, 3, 4 and 5, as well as sub ranges such as 2-5, 3-5, 2-3, 2-4, 1-4, etc.

The term “substantially free” or “essentially free” as used herein means that there is less than about 2% by weight of a specific material added to a composition, based on the total weight of the compositions. Nonetheless, the compositions may include less than about 1 wt. %, less than about 0.5 wt. %, less than about 0.1 wt. %, or none of the specified material.

All ranges and values disclosed herein are inclusive and combinable. For examples, any value or point described herein that falls within a range described herein can serve as a minimum or maximum value to derive a sub-range, etc.

All publications and patent applications cited in this specification are herein incorporated by reference, and for any and all purposes, as if each individual publication or patent application were specifically and individually indicated to be incorporated by reference. In the event of an inconsistency between the present disclosure and any publications or patent application incorporated herein by reference, the present disclosure controls.

1. A hair treatment composition comprising:
(a) one or more polycarboxylic acid and/or a salt thereof;
(b) one or more amino acid, and/or a salt thereof; and
(c) one or more antioxidant comprising an aromatic ring having two alcohol functional groups.

2. The hair treatment composition of claim 1 comprising one or more color altering component.

3. The hair treatment composition of claim 2, wherein the one or more color altering component is selected from one or more of: a bleaching agent; a developer; a colorant selected from oxidative dye precursors and direct dyes; a bleach and a developer; a colorant selected from oxidative dye precursors and direct dyes and a developer; and mixtures thereof.

4. The hair treatment composition of claim 2, wherein the hair treatment composition and one or more color altering component are premixed so that the hair treatment composition is built-in the one or more color altering component.

5. The hair treatment composition of claim 1, wherein the one or more antioxidant comprises a flavonoid glycoside.

6. The hair treatment composition of claim 1, wherein the one or more antioxidant comprises baicalin.

7. The hair treatment composition of claim 1, wherein the one or more antioxidant comprises at least one polyphenol selected from baicalin, ferulic acid, pine bark extract, polydatin, ellagic acid and/or mixtures thereof.

8. The hair treatment composition of claim 1, wherein the one or more polycarboxylic acid comprises dicarboxylic acids, one or more tricarboxylic acids, a salt thereof, and/or a mixture thereof.

9. The hair treatment composition of claim 8, wherein the one or more dicarboxylic acids are selected from oxalic acid, malonic acid, malic acid, glutaric acid, citraconic acid, succinic acid, adipic acid, tartaric acid, fumaric acid, maleic acid, sebacic acid, azelaic acid, dodecanedioic acid, phthalic acid, isophthalic acid, terephthalic acid, 2,6-naphthalene dicarboxylic acid, a salt thereof, and a mixture thereof; and the one or more tricarboxylic acids are selected from the group consisting of citric acid, isocitric acid, trimesic acid, aconitric acid (cis and/or trans), propane-1,2,3-tricarboxylic acid, a salt thereof, and a mixture thereof.

10. The hair treatment composition of claim 1, wherein the one or more polycarboxylic acid comprises dicarboxylic acids selected from maleic acid, malonic acid, citric acid, a salt thereof, and/or a mixture thereof.

11. The hair treatment composition of claim 1, wherein the one or more amino acid comprises one or more aliphatic amino acid selected from glycine, alanine, valine, leucine, isoleucine, a salt thereof, and a mixture thereof.

12. The hair treatment composition of claim 11, wherein the one or more amino acid comprises glycine and/or a salt thereof.

13. The hair treatment composition of claim 1 comprising one or more cationic conditioning agent, wherein the one or more cationic conditioning agent is selected from one or more monoalkyl quaternary amines, dialkyl quaternary amines, polyquaternium compounds, or a mixture thereof.

14. The hair treatment composition of claim 1 comprising one or more oils selected one or more ester oils, mineral oil, silicone oils, fluoro oils, vegetable oils, animal oils, hydrocarbon-based oils, and mixtures thereof.

15. The hair treatment composition of claim 1 comprising one or more thickening agents selected from carboxylic acid polymer(s), crosslinked polyacrylate polymer(s), polyacrylamide polymer(s), polysaccharide(s), gum(s), and mixtures thereof.

16. The hair treatment composition of claim 15, wherein the thickening agent is guar gum.

17. A hair lightening composition comprising:
(a) one or more polycarboxylic acid and/or a salt thereof;
(b) one or more amino acid, and/or a salt thereof;
(c) one or more antioxidant comprising an aromatic ring having two alcohol functional groups;
(d) one or more bleaching agent.

18. The hair lightening composition of claim 17, wherein the one or more antioxidant comprises a baicalin.

19. The hair lightening composition of claim 17, wherein the one or more antioxidant is premixed in the hair lightening composition so it is built-in to the hair lightening composition.

20. The hair lightening composition of claim 17, wherein the one or more polycarboxylic acid comprises dicarboxylic acids selected from maleic acid, malonic acid, citric acid, a salt thereof, and/or a mixture thereof.

21. The hair lightening composition of claim 17, wherein the one or more amino acids comprises one or more aliphatic amino acids selected from glycine, alanine, valine, leucine, isoleucine, a salt thereof, and a mixture thereof.

22. A hair lightening composition comprising:
(a) about 0.1 to about 5 wt. % of one or more polycarboxylic acid selected from maleic acid, malonic acid, citric acid, a salt thereof, or a mixture thereof;
(b) about 0.01 to about 5 wt. % of one or more aliphatic amino acid selected from glycine, alanine, valine, leucine, isoleucine, a salt thereof, and a mixture thereof;
(c) about 0.01 to about 5 wt. % of one or more antioxidant comprising an aromatic ring having two alcohol functional groups, optionally selected from baicalin;
(d) about 10 to about 80 wt. % of one or more persulfates selected from the group consisting of sodium persulfate, potassium persulfate, ammonium persulfate, and a mixture thereof;
(e) about 0.1 to about 10 wt. % of one or more oils;
(f) about 0.1 to about 10 wt. % of one or more thickening agents;
(g) about 0.1 to about 10 wt. % of one or more anionic surfactants; and
(h) about 1 to about 40 wt. % of one or more alkalizing agents;
wherein the wt. % in all instances is based on the total weight of the hair lightening composition.

23. A kit comprising:
i. a hair lightening composition comprising:
(a) one or more polycarboxylic acid and/or a salt thereof;
(b) one or more amino acid, and/or a salt thereof;
(c) one or more antioxidant comprising an aromatic ring having two alcohol functional groups, optionally comprising baicalin;
(d) one or more bleaching agent;
ii. an aqueous developer composition comprising one or more peroxides; and
iii. optionally, a third composition comprising one or more hair conditioning agents and/or one or more direct dyes.

24. A method for lightening hair comprising:
a. mixing a hair lightening composition with an aqueous developer composition comprising one or more peroxides, the hair lightening composition comprising:
i. one or more polycarboxylic acid and/or a salt thereof;
ii. one or more amino acid, and/or a salt thereof;
iii. one or more antioxidant comprising an aromatic ring having two alcohol functional groups, optionally comprising baicalin;
iv. one or more bleaching agent;
b. applying the mixture onto hair and allowing the mixture to remain on the hair for about 1 to about 45 minutes; and
c. rinsing the mixture from hair.
